(19) United States
(12) Patent Application Publication
(10) Pub. No.: US 2018/0142307 A1
(43) Pub. Date: May 24, 2018

(54) RECORDING AND MAPPING LINEAGE INFORMATION AND MOLECULAR EVENTS IN INDIVIDUAL CELLS

(71) Applicant: California Institute of Technology, Pasadena, CA (US)

(72) Inventors: Long CAI, Pasadena, CA (US); Michael B. ELOWITZ, Pasadena, CA (US); James D. LINTON, Pasadena, CA (US); Joonhyuk CHOI, Pasadena, CA (US); Kirsten L. FRIEDA, Pasadena, CA (US); Sahand HORMOZ, Pasadena, CA (US); Ke-Huan Kuo CHOW, Pasadena, CA (US)

(21) Appl. No.: 15/713,597

(22) Filed: Sep. 22, 2017

Related U.S. Application Data
(63) Continuation-in-part of application No. 14/620,133, filed on Feb. 11, 2015, now abandoned.
(60) Provisional application No. 61/938,490, filed on Feb. 11, 2014.

(51) Int. Cl. C12Q 1/6888; G06F 19/14

(52) U.S. Cl. CPC C12Q 1/6888; C12Q 2600/156; G06F 19/14

(57) ABSTRACT

Methods and systems for recording and mapping lineage information and molecular events in individual cells are provided. Molecular changes, which may result from random or specific molecular events, are introduced to defined regions in cells over multiple cell cycle generations. Techniques such as fluorescent imaging are applied to track and identify the molecular changes before such information is used for lineage analysis or for identifying key processes and key players in cellular pathways.

### CROSS-REFERENCE TO RELATED APPLICATIONS

This application claims priority from U.S. patent application Ser. No. 14/620,133, filed Feb. 11, 2015 and entitled “Recording and Mapping Lineage Information and Molecular Events in Individual Cells,” which in turn claims priority to U.S. Provisional Patent Application No. 61/938,490, filed on Feb. 11, 2014, each of which is incorporated herein by reference in its entirety.

### FIELD OF THE INVENTION

The invention disclosed herein generally relates to methods and systems for creating or triggering molecular changes (e.g., genetic mutations or modification) in defined regions in a genome. In particular, the invention disclosed herein relates to the design and characteristics of such defined regions and methods and systems for creating or triggering molecular changes that lead to or result from certain random or specific molecular events such as signal transduction. Further, the invention disclosed herein relates to methods and systems for capturing, characterizing and analyzing the molecular changes, in order to extrapolate lineage or phylogenetic information connecting such molecular events or record the history of cellular events.

### BACKGROUND

A fundamental problem throughout developmental biology is determining the lineages through which cells differentiate to form tissues and organs. Lineage information is critical for addressing basic developmental questions in diverse systems including the brain and tumor genesis. Although the lineage map of embryonic development in C. elegans was worked out three decades ago, systematic techniques that can produce such comprehensive maps in more complex organisms are lacking. Furthermore, in order to understand how lineages are determined, the lineage tree needs to be connected directly to the molecular changes and eventually molecular events that occur in cells to determine developmental decisions.

Existing lineage determination approaches have severe limitations. Most current approaches are based on marking the descendants of selected cells. Site-specific recombinases such as FLP and Cre can be used to mark the descendants of particular cells. More sophisticated variants, such as Brainbow, can mark many distinct cells at one time to follow their descendants. However, these techniques do not allow one to follow multiple lineage decisions or reconstruct an entire tree in a single experiment. Finally, no existing technique enables one to systematically record the molecular events that occur during lineage determination within the cells themselves.

What is needed in the art are vastly improved tools for tracking lineage information, capturing molecular changes during development and reading out this information with minimal perturbations to cells and organisms, ideally within the cells themselves.

### SUMMARY OF THE INVENTION

In one aspect, provided herein is a method for characterizing lineage information or recording molecular events among cells in a cell population. The method comprises the steps of: introducing, over a time period of multiple cell cycle generations, a plurality of molecular changes in at least one of one or more genetic scratchpads in one or more cells in a cell population, characterizing, at one or more time points during the time period, a status of molecular changes at each time for the plurality of target sites in each genetic scratchpad in cells in the cell population, wherein the cells are essentially intact or undisrupted, wherein at least one time point in the one or more time points is two or more cell cycle generations from the beginning of the time period; and establishing lineage connections between cells from different cell cycle generations by comparing statuses of molecular changes of the cells.

In some embodiments, the cell population comprises cells that have developed for one or more cell cycle generations. In some embodiments, each genetic scratchpad in the one or more genetic scratchpads comprises a polynucleotide sequence and a plurality of target sites within the polynucleotide sequence. In some embodiments, each of the plurality of mutations is associated with a target site among the plurality of target sites. In some embodiments, the molecular changes represent one or more molecular events: they are either the cause or result of one or more molecular events.

In some embodiments, characterizing step further comprises the steps of applying a set of probes to the cell population and characterizing the mutation status in a plurality of cells in the cell population by detecting the presence or absence of visible signals in the plurality of cells.

In some embodiments, each probe in the set recognizes and binds to a corresponding target sequence in a target site among the plurality of target sites.

In some embodiments, each probe comprises a label that produces a visible signal upon binding between the probe and its unique target sequence.

In some embodiments, each target site comprises a guide sequence that is recognized by a unique guide molecule, and wherein binding of the unique guide molecule to the guide sequence recruits a molecule that is capable of creating a mutation at the target site.

In some embodiments, the guide sequence comprises a nucleotide sequence having a length between about 15 nucleic acids to about 80 nucleic acids. In some embodiments, the guide sequence comprises a nucleotide sequence having a length between about 15 nucleic acids to about 30 nucleic acids.

In some embodiments, the unique guide molecule is a guide RNA (gRNA).

In some embodiments, the molecule is a nuclease, recombinase or integrase. In some embodiments, the nuclease is Cas9 nuclease

In some embodiments, the multiple time points during the time period cover two or more cell cycle generations. In some embodiments, the multiple time points during the time period cover three or more cell cycle generations. In some embodiments, the multiple time points during the time period cover five or more cell cycle generations.

In some embodiments, the plurality of molecular changes comprises a plurality of mutations. In some embodiments, the plurality of mutations comprises one selected from the group consisting of an insertion mutation, a deletion mutation, a point mutation, multiple point mutations, and combinations thereof.

In some embodiments, each target site further comprises a barcode sequence linked to the guide sequence.

In some embodiments, the barcode sequence comprises a nucleotide sequence having a length between about 400 nucleic acids to about 2,000 nucleic acids. In some embodiments, the barcode sequence nucleic acids a nucleotide sequence having a length between about 50 nucleic acids to about 200 nucleic acids.

In some embodiments, each target site in a plurality of target sites within at least one genetic scratchpad comprises the same guide sequence that is recognized by a unique guide molecule.

In some embodiments, each target site in a plurality of target sites within at least one genetic scratchpad comprises a different guide sequence that is recognized by a unique and different guide molecule.

In some embodiments, the plurality of target sites within at least one genetic scratchpad comprises one selected from the group consisting of two or more different guide sequences, three or more different guide sequences, five or more different guide sequences, eight or more different guide sequences, 10 or more different guide sequences, 15 or more different guide sequences, 20 or more different guide sequences, and 30 or more different guide sequences.

In some embodiments, the characterizing step further comprises the steps of: applying a set of probes to cells in the cell population and characterizing a mutation status at the plurality of target sites based on the absence and presence of signals.

In some embodiments, each probe comprises a nucleic acid sequence designed to bind to a target site within the plurality of target site. In some embodiments, each probe is associated with a label that produces a signal upon binding between the probe and its corresponding target site.

In some embodiments, absence of a signal indicates a mutation at the target site and the presence of a signal indicates an intact target site, or vice versa

In some embodiments, the set of probes comprises RNA probes or DNA probes. In some embodiments, probes in the set of probes are associated with multiple labels that produce different signals.

In some embodiments, each probes of the set of probes are designed to bind to a guide sequence within a target site within the plurality of target site.

In some embodiments, each probes of the set of probes are designed to further bind to a barcode sequence linked to the guide sequence within a target site within the plurality of target site.

In one aspect, provided herein is a system for characterizing lineage information or recording molecular events among cells in a cell population. The system comprises a few components, including for example, a housing component, a characterization component and an analytical component.

In some embodiments, the housing component provides housing for one or more cells in a cell population. A plurality of molecular changes is introduced over a time period of multiple cell cycle generations in at least one of one or more genetic scratchpads in one or more cells in a cell population. In some embodiments, the cell population comprises cells that have developed for one or more cell cycle generations. In some embodiments, each genetic scratchpad in the one or more genetic scratchpads comprises a polynucleotide sequence and a plurality of target sites within the polynucleotide sequence. In some embodiments, each of the plurality of molecular changes is associated with a target site among the plurality of target sites.

In some embodiments, the characterization component is configured to characterize the cell population. At one or more time points during the time period, a status of molecular changes at each time for the plurality of target sites in each genetic scratchpad in cells in the cell population is characterized, for example, by fluorescence imaging techniques using probes that recognize mutations with target sites in genetic scratchpads in cells in the cell population. In some embodiments, the molecular changes represent one or more molecular events: they are either the cause or result of one or more molecular events.

As disclosed herein, molecular changes include any changes that are reflected at the genetic level (e.g., at the RNA transcription level) can be detected and/or quantified by the method disclosed herein. For example, RNA can be turned on and off in response to certain conditions: tumorigenesis often correlates with the overexpression of one or more genes.

In some embodiments, the cells are essentially intact or undisrupted, wherein at least one time point in the one or more time points is two or more cell cycle generations from the beginning of the time period.

In some embodiments, the analytical component is designed to receive data from the characterization component. The analytical components establish lineage connections between cells from different cell cycle generations by comparing mutation statuses of the cells.

Without any limitation, embodiments disclosed herein can be applied to any aspect of the invention, alone or in any combinations.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 depicts an exemplary process.

FIG. 2A depicts an exemplary embodiment of a scratchpad design.

FIG. 2B depicts an exemplary embodiment of a scratchpad design with guide RNA (gRNA) binding sequences.

FIG. 2C depicts an exemplary embodiment of a scratchpad design with guide RNA (gRNA) binding sequences and barcode sequences.

FIG. 2D depicts an exemplary embodiment of a target site within a genetic scratchpad.

FIG. 3A depicts the mechanism for a Clustered Regularly Interspaced Short Palindromic Repeats (CRISPR) system.

FIG. 3B depicts an exemplary expression cassette for gRNA expression.

FIG. 3C depicts an exemplary expression cassette for Cas9 protein expression.

FIG. 4A depicts an exemplary embodiment with multiple gRNAs.

FIG. 4B depicts an exemplary embodiment of a genetic scratchpad with multiple gRNA binding regions.

FIG. 4C depicts an exemplary embodiment, illustrating mutations in multiple cell cycle generations.

FIG. 4D depicts an exemplary embodiment with a single gRNA.

FIG. 4E depicts an exemplary embodiment of a genetic scratchpad with a gRNA binding region coupled with multiple barcode sequences.

FIG. 4F depicts an exemplary embodiment, illustrating mutations in multiple cell cycle generations.

FIG. 5A depicts an exemplary embodiment, illustrating multiple rounds of probe hybridization.

FIG. 5B depicts exemplary schematic images from multiple rounds of probe hybridization.

FIG. 5C depicts exemplary embodiments, illustrating the color code representing a particular target site.

FIG. 6A depicts an exemplary embodiment with multiple gRNAs.

FIG. 6B depicts an exemplary embodiment, illustrating multiple genetic scratchpads each containing one of a few distinct gRNA binding region.

FIG. 6C depicts an exemplary embodiment, illustrating mutations in multiple cell cycle generations.

FIG. 7A depicts an exemplary embodiment of a genetic scratchpad.

FIG. 7B depicts an exemplary linage tree.

FIGS. 7C-7E illustrate an example overall system for recording and in situ readout of cell lineage. 7C) Barcoded scratchpads provide a general purpose recording element whose state can be irreversibly altered by Cas9/gRNA-mediated cleavage. 7D) The recording system consists of three types of components, all stably integrated into the genome: (1) a Cas9 variant containing an inducible degron (DD) that is stabilized by the small molecule Shield1. (2) A Wnt-inducible gRNA targeting the scratchpad, co-expressed with a fluorescent protein (mTurquoise). Ribozyme sequences (HH, HDV) enable gRNA excision. (3) A set of barcoded scratchpads (two-colour elements) integrated throughout the genome. Inverted triangles in 7C and 7D denote PiggyBac terminal repeats, used for genome integration. 7E) The recording and readout process. During recording, scratchpads collapse stochastically as cells proliferate, producing distinct scratchpad states in each cell. During readout, individual mRNA molecules are detected with a single scratchpad-specific probe set (orange, inset), and multiple barcode-specific probe sets (blue, green, inset) through sequential rounds of hybridization and imaging. Uncollapsed scratchpads produce co-localized barcode and scratchpad signals (overlapping dots), while collapsed scratchpads produce only a barcode-specific signal (single dots).

FIG. 8A depicts an exemplary embodiment, illustrating deletion mutation in a genetic scratchpad in mammalian cells. Additional examples of deletions within a scratchpad are illustrated in FIG. 17.

FIG. 8B depicts an exemplary embodiment, illustrating deletion mutation in a genetic scratchpad in yeast cells.

FIG. 9 depicts an exemplary embodiment, showing the effects of mismatched gRNAs.

FIG. 10A depicts an exemplary embodiment, showing single molecular fluorescence in situ hybridization (smFISH) image detection of genetic scratchpad in mammalian cells.

FIG. 10B depicts an exemplary embodiment, showing smFISH image detection of genetic scratchpad in yeast cells.

FIG. 11A depicts an exemplary embodiment, showing smFISH image detection of genetic mutation within genetic scratchpad in mammalian cells.

FIG. 11B depicts an exemplary embodiment, showing smFISH image detection of genetic mutation within genetic scratchpad in mammalian cells.

FIG. 12A depicts an exemplary embodiment, showing snapshots of single cells with genetic scratchpads dividing over time.

FIG. 12B depicts an exemplary embodiment, showing smFISH image detection of genetic mutation within genetic scratchpad in mammalian cells.

FIG. 12C depicts an exemplary lineage tree.

FIG. 13 depicts an exemplary embodiment, illustrating barcoding in cells.

FIG. 14A depicts an exemplary embodiment, illustrating computer-simulated mutations over multiple generations.

FIG. 14B depicts an exemplary embodiment, illustrating a lineage constructed based on the computer-simulated mutation data from FIG. 14A.

FIGS. 15A-15E depict in situ readout of scratchpad state. 15A), smFISH readout of scratchpad state in two cells (white outlines). The scratchpad associated with barcode 2 has collapsed in the lower cell, but remains uncollapsed in the upper cell. Overlaid images are slightly offset for visual clarity. 15B), Histograms of scratchpad smFISH signal intensities, identified as collapsed (blue) or uncollapsed (orange) based on scratchpad-barcode co-localization. The fraction of collapsed scratchpads increased after 48 h of activation (top versus bottom panel). Far right bars indicate smFISH signal exceeding the maximum displayed intensity. 15C), Scratchpad collapse accumulates over time post activation. Box plots show median (red bar), first and third quartiles (box) and extrema for four highly expressed barcodes; n=1,826, 418, 610, 545 cells, left to right. Activated samples in b and c only include gRNA-expressing cells, as measured by co-expression of mTurquoise. 15D), Multiplexed readout of barcoded scratchpads (scratchpad, SP; barcode, BC) by sequential rounds of hybridization with distinct probe sets (colors) provide information about the collapse status of multiple barcoded scratchpads in each cell (right). 15D, Example of seqFISH analysis. Scratchpads (red) and three pairs of barcodes (middle images) are shown (pseudo-colored). Solid and dashed circles at barcode positions indicate uncollapsed and collapsed scratchpads, respectively. Barcode data are superimposed on the scratchpad image in the final panel. For clarity, additional hybridizations and barcodes are not shown. Scale bars (15A, 15E), 10 μm (left images) and 2 μm (magnified panels). FIG. 15 relates to FIG. 5: FIG. 5 provides a schematic representation that corresponds to the experimental data depicted in FIG. 15.

FIGS. 16A through 16C illustrates an exemplary schematic for cellular event recording. 16A), gRNA1 (orange) is constitutively expressed for lineage reconstruction, while the orthogonal gRNA2 (purple) and gRNA3 (green) are expressed in response to specific signals and target independent scratchpads sets. 16B), schematic showing recording of possible signaling histories (purple and green shading indicate periods when signals 1 and 2, respectively, are present. g, Reconstruction of simulated event histories in a six-generation tree. The signals recorded along two branches (yellow) are shown (bottom panels), including the actual simulated signals (thick lines), examples of individual reconstructed signals (dashed lines), and the average reconstructed signals (solid lines; mean±s.d., n=500 trees). FIG. 16 is similar to FIG. 6.

FIGS. 17A through 17G illustrate how barcoded scratchpads collapse to truncated products in activated cells and are stable in full-length and collapsed forms. 17A), Agarose gel electrophoresis of PCR amplified scratchpads reveals scratchpad collapse after gRNA induction. Full-length scratchpads were amplified from plasmid DNA (lane 1), as well as from cells without gRNA constructs (lane 3), or with uninduced gRNAs (lane 4). By contrast, cells expressing gRNA showed shorter products (lane 5). Cells with no scratchpads are also shown as a negative control (lane 2). Bands corresponding to the full-length scratchpad and the collapsed scratchpad are indicated (arrows). Note that the laddering effect seen in all lanes and gels is due in part to PCR amplification artefacts with the repetitive arrays. 17B), The lowest molecular weight band from scratchpad collapse, as shown in lane 5 in a, was extracted and subcloned into a vector. Nine of the colonies were sequenced. They aligned to a single repeat unit with 5′ and 3′ flanking regions, suggesting complete collapse of the repeats owing to Cas9 activity. Six of the nine sequencing reads resulted in collapse to a perfect single repeat (with a possible point mutation in the scratchpad sequence associated with barcode 2), and the remaining three sequencing reads had additional small deletions in the scratchpad. 17C), Scratchpad collapse requires induction of both Cas9 and gRNA. The gel shows scratchpad states for MEM-01 cells treated with no ligand, with Shield1 (to stabilize Cas9 protein), with Wnt3a (to induce gRNA expression), and with both Wnt3a (100 ng per ml) and Shield1 (100 nM), all after 48 h. 17D), Scratchpad collapse increased with increasing gRNA activation, as assessed using smFISH to detect scratchpad co-localization with four highly expressed barcodes. Cells were analyzed either without gRNA activation or 48 h after gRNA activation by addition of Wnt3a and Shield1 (same concentrations as in 17C). gRNA expression was measured by the intensity of co-expressed nuclear mTurquoise signal. Box plots show median (red bar), first and third quartiles (box), and extrema of distributions; n=1,826; 1,081; 345; 191 cells, left to right. Related to FIG. 15C and in 17E-17G, Scratchpad states remain stable over extended periods. 17E), Unactivated MEM-01 cells maintained uncollapsed scratchpads over timescales of months. 17F), To check the stability of individual barcoded scratchpad variants over time, multiple subclones of MEM-01 were isolated after no activation (control; top panels) and after a pulse of activation for 24 h (Wnt3a 100 ng per ml, Shield1 100 nM; bottom panels). Subclones were assessed for the states of different barcoded scratchpad types after initial isolation (0 month relative age, left) and after one month of maintenance (right). The apparent collapse states (from uncollapsed to fully collapse) of the barcoded scratchpad types were distinct in different subclones and remained stable over a month, indicating that scratchpad states are stable over these timescales. 17G), Barcoded scratchpads are also stable over long periods as assessed by smFISH readout. The fraction per cell of barcode transcripts (from four distinct barcode types) that co-localized with scratchpad signal was essentially unchanged between an unactivated low passage cell culture and one maintained for over a month. The imperfect co-localization fraction is largely the result of errors in smFISH detection and not gradual scratchpad collapse. Boxplots as in 17D; n=1,826, or 983 cells, left to right.

FIGS. 18A through 18F depict an example showing lineage reconstruction in ES cell colonies. 18A), Time-lapse videos of colony growth were acquired to provide lineage ‘ground truth’ (dashed lines) for later validation of reconstructed lineages, but not for reconstruction itself. 18B), At the end of the movie, seqFISH was performed, as in FIG. 15. Scale bar, 20 μm. 18C), Examples of how barcoded scratchpad collapse patterns reflect cell lineage. 18D), Sample readout for the colony in 18A-18C, showing the number of barcode transcripts detected (bubble size) and the un-collapsed fraction (color scale). 18E), Data from 18D were used to compute a matrix of cell-to-cell barcode ‘distance’ (dissimilarity) scores. 18F), reconstructed lineage tree for the same colony. Percentages on the tree represent the frequencies of clade occurrence from a barcode resampling bootstrap procedure. In this case, the reconstructed tree matches that obtained from the video. The data presented in FIG. 18 provides further illustration to FIG. 12.

### DETAILED DESCRIPTION OF THE INVENTION

Unless otherwise noted, terms are to be understood according to conventional usage by those of ordinary skill in the relevant art.

As used herein, the term “an essentially intact or undisrupted cell” refers to a cell that is completely intact or largely conserved with respect to its macromolecular cellular content. For example, a cell within the meaning of this term can include a cell that is made at least partially permeable such that external buffer and reagents can be introduced into the cell. Such external reagents include but are not limited to probes, labels, labeled probes, and/or combinations thereof.

As used herein, the term “genetic scratchpad” refers to a polynucleotide sequence within a prokaryotic or eukaryotic cell. In some embodiments, the genetic scratchpad can be synthesized in vitro and then put into the cell. In some embodiments, the genetic scratchpad refers to a defined location within the natural genomic sequence of the cell. In some embodiments, the genetic scratchpad can refer to a defined location within the natural genomic sequence of the cell that has been modified. Within the polynucleotide sequence of a genetic scratchpad, there are multiple target sites. In some embodiments, each target site comprises a guide sequence that can be recognized by a unique guide molecule.

As use herein, the term “molecular event” refers to occurrences that happen in a cell and that we can record with our method, like a signaling event, transcription factor activity or even a more complex process such as tumor genesis or kinase transduction pathway. The term “molecular change” or “molecular alteration or mutation” refers to a change that occurs in the scratchpad, like a genetic mutation or genetic modification. The molecular change can be the result or the cause of a molecular event.

As used herein, the term “mutation” or “genetic mutation” refers to any recognizable variation in nucleotide sequence that can be used in accordance with the present invention. For example, a mutation can be a deletion or an insertion of a polynucleotide sequence. In some embodiments, the absence or presence of the polynucleotide sequence can be indicated by using one or more visible indicia; for example, a nucleotide hybridization probe with a fluorescent color label. The length of the polynucleotide deletion or insertion can vary with applications and sensitivities of the probes. For example, the polynucleotide comprises 10 or fewer nucleic acids, 20 or fewer nucleic acids, 30 or fewer nucleic acids, 40 or fewer nucleic acids, 50 or fewer nucleic acids, 60 or fewer nucleic acids, 70 or fewer nucleic acids, 80 or fewer nucleic acids, 90 or fewer nucleic acids, 100 or fewer nucleic acids, 150 or fewer nucleic acids, 200 or fewer nucleic acids, 250 or fewer nucleic acids, 300 or fewer nucleic acids, 350 or fewer nucleic acids, 400 or fewer nucleic acids, 450 or fewer nucleic acids, 500 or fewer nucleic acids, 600 or fewer nucleic acids, 700 or fewer nucleic acids, 800 or fewer nucleic acids, 900 or fewer nucleic acids, 1,000 or fewer nucleic acids, 1,500 or fewer nucleotides, 2,000 or fewer nucleic acids, 5,000 or fewer nucleic acids, or 10,000 or fewer nucleic acids. In some embodiments, the polynucleotide insertion or deletion is longer than 10,000 nucleic acids.

As used herein, the term “guide sequence” refers to a sequence within a target site that can be recognized by a molecule or set of molecules that create or trigger molecular changes such as genetic mutations or modifications that lead to certain molecular events such as signal transduction, tumor genesis or metastasis, and etc. Alternatively, molecular events can be the cause of certain molecular changes. This guide molecule may be a guide RNA (gRNA), which recruits a second molecule such as nuclease to the binding site to create mutations. In some embodiments, a guide sequence comprises 10 or fewer nucleic acids, 20 or fewer nucleic acids, 30 or fewer nucleic acids, 40 or fewer nucleic acids, 50 or fewer nucleic acids, 60 or fewer nucleic acids, 70 or fewer nucleic acids, 80 or fewer nucleic acids, 90 or fewer nucleic acids, 100 or fewer nucleic acids, 150 or fewer nucleic acids, or 250 or fewer nucleic acids. In some embodiments, the guide sequence comprises 500 or more nucleic acids or even 1,000 nucleic acids when tandem gRNAs are implemented in a target site.

As used herein, the term “barcode” refers to a sequence within a target site that can be used to identify the particular target site. A barcode sequence is also referred to as a target sequence. In some embodiment a barcode sequence can be any sequence that uniquely identifies the associated scratchpad. In some embodiments, a barcode sequence is linked to a corresponding guide sequence. In some embodiments, a barcode sequence comprises 10 or fewer nucleic acids, 20 or fewer nucleic acids, 30 or fewer nucleic acids, 40 or fewer nucleic acids, 50 or fewer nucleic acids, 60 or fewer nucleic acids, 70 or fewer nucleic acids, 80 or fewer nucleic acids, 90 or fewer nucleic acids, 100 or fewer nucleic acids, 150 or fewer nucleic acids, 250 or fewer nucleic acids, 500 or fewer nucleic acids, 1,000 or fewer nucleic acids, 1,500 or fewer nucleic acids, 2,000 or fewer nucleic acids, or 5,000 or fewer nucleic acids. In some embodiments, a barcode sequence comprises more than 5,000 nucleic acids.

As used herein, the term “probe” refers to any composition that can be specifically associated with a target nucleotide within a cell. A probe can be a small molecular or a large molecule. Exemplary probes include but are not limited to nucleic acids such as oligos. In some embodiments, a probe is associated with a visible label such as a fluorescence label to indicate the presence of a certain nucleotide sequence. In some embodiments, the probe can be a DNA probe or an RNA probe. In some embodiments, a probe sequence comprises 10 or fewer nucleic acids, 20 or fewer nucleic acids, 30 or fewer nucleic acids, 40 or fewer nucleic acids, 50 or fewer nucleic acids, 60 or fewer nucleic acids, 70 or fewer nucleic acids, 80 or fewer nucleic acids, 90 or fewer nucleic acids, 100 or fewer nucleic acids, 150 or fewer nucleic acids, 250 or fewer nucleic acids, or 500 or fewer nucleic acid. In some embodiments, a probe comprises more than 500 nucleic acids.

As used herein, the term “label” refers to any composition that can be used to generate the signals that constitute an indicium. The signals generated by a label can be of any form that can be resolved subsequently to constitute the indicium. Preferably, the signal is a light within the visible range. However, it will be understood by one of skill in the art that equipment and devices are available for recording and monitoring light of any wavelength. The label can also constitute any moiety, such as a hapten, that can be recognized by an antibody. This secondary antibody can be conjugated to a fluorescent molecule or an enzyme that can produce signals that constitute an indicium.

Disclosed herein are methods and systems for capturing molecular events within cells to extrapolate lineage information between cells from different generations. An exemplary system includes one or more of the following components: one or more genetic scratchpad(s) where molecular changes such as genetic mutations or modification will occur; a writing component for creating the genetic mutations within the genetic scratchpad; a characterization component for capturing the mutation status of a genetic scratchpad by identifying the presence and absence of such genetic mutations; and an analysis component for reading out mutations that have been created in the scratchpads.

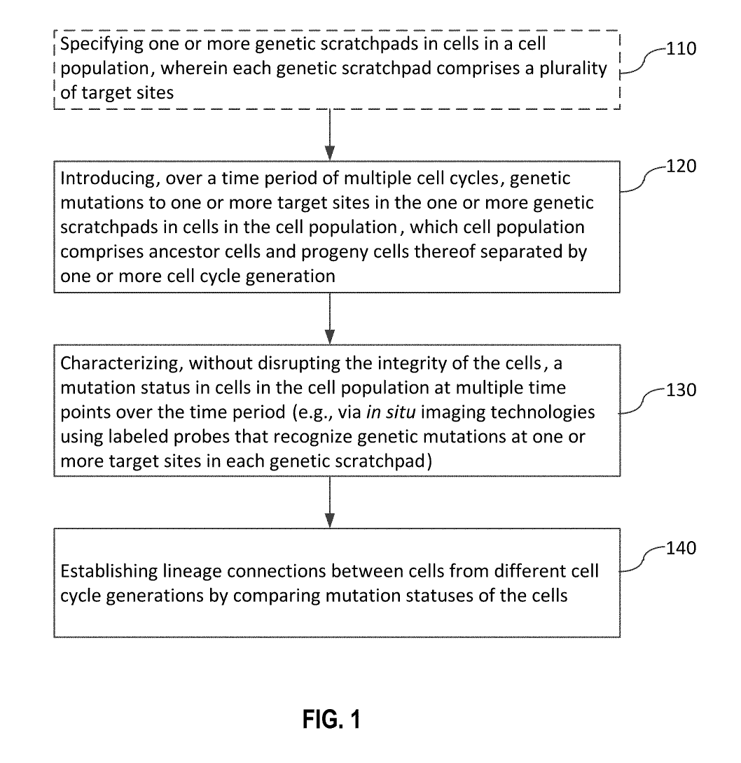

FIG. 1 outlines an exemplary process disclosed herein.

At step 110, one or more genetic scratchpads are specified with a cell. As noted above, molecular changes as disclosed herein (e.g., genetic mutations or modification) take place within the genetic scratchpads. More precisely, a genetic scratch comprises one or more target sites and the molecular changes take place at the target sites. One of skill in the art will understand that similar molecular changes also occur elsewhere inside the cells. However, those events are not within the scope of subsequent analysis. In addition, after the molecular changes have taken place, subsequent analysis (such as visualization of the presence and absence of genetic mutations) will also be focused on the genetic scratchpad, for example at the target sites. As disclosed herein, the terms “genetic scratchpad,” “scratchpad” and variations thereof are used interchangeably.

As disclosed herein, a genetic scratchpad comprises nucleotide sequences that are synthesized in vitro. Alternatively, a genetic scratchpad comprises a natural region of the genomic sequence of the cell. Still alternatively, a genetic scratchpad comprises a hybrid of synthetic and natural sequences. Still alternatively, a genetic scratchpad comprises natural nucleotide sequence that has been modified at one or more locations.

At step 120, molecular changes such as genetic mutations are introduced into one or more genetic scratchpads over a time period that spans multiple cell cycle generations. Such molecular changes can be genetic mutations such as insertions or deletions of nucleotide sequences at one or more of the target sites within a genetic scratchpad. Alternatively, the molecular changes can be genetic modifications. For example, a DNA segment can be methylated to alternative its functionality or possibility of be transcribed. In particular, a methyl-transferase can be fused to cas9 and target specific sites to bring about changes in a target site in one or more genetic scratchpads.

At any given cell cycle, the same molecular changes can be introduced into multiple genetic scratchpads or multiple target sites within the same scratchpad. In some embodiments, no molecular changes take place in any genetic scratchpad during a particular cell cycle.

At step 130, the genetic status of the genetic scratchpads (e.g., the status of target sites within the scratchpads) within cells from step 120 is characterized. Characterization of genetic status includes identifying the presence and absence of genetic mutations at target sites within one or more scratchpads.

In some embodiments, labeled probes designed to bind specific sequences in the target sites are used. For example, an intact target site (e.g., no molecular change has taken place at the site) will allow proper binding between the labelled probes and the target site. Upon binding, the label can be induced to emit signals such as fluorescent light. In contrast, if a target site is disrupted by a molecular change, for example, due to deletion or insert of nucleotide sequences, a probe specifically targeting the site will no longer be able to bind. Consequently, there will be no label attached to the target site and no subsequent fluorescent signals. In exemplary embodiments, the presence of fluorescent signal at a target site suggests that no molecular changes have occurred while absence of such a signal at a target site suggests that one or more molecular changes have occurred to disrupt the sequence at the target site. In alternate embodiments, the induced mutation could result in the emergence of a new, detectable fluorescence signal. For example, in the absence of a mutation, fluorescent probes might not bind the target site. After a particular mutation, such as an insertion mutation, probes will be able to bind the site and produce a detectable signal.

Over multiple cell cycles, a cell (e.g., an ancestor cell) at the beginning of the time period has divided into multiple progeny cells. As such, at a given time point, there are progeny cells present that carry information about their past and ancestry. As disclosed herein, characterization of genetic status is carried out for cells in the cell population at a defined time point. Genetic status characterization of cells within the population allows construction of their lineage relationships as well as a record of any other historical events being tracked. The characterization time point is selected to provide information across the time window of interest, which ideally spans multiple cell cycle generations to allow reconstruction of a comprehensive history.

Alternatively, characterization can also be carried out at multiple, distinct time points. The time points can be chosen as desired to focus on changes across cell generations of interest. In some embodiments, this can be helpful in order to effectively sample changes across long processes and/or focus on multiple subsets of events within these processes: for example, for extracting lineage information and cellular histories during stereotypic, developmental processes, where defined cell types emerge at distinct times.

In some embodiments, presence and absence of fluorescent signals are determined by comparing images of both ancestor and progeny cells.

Here, the genetic status of a given cell is assessed while the structural and functional integrity within the cell is maintained. Additionally minimal perturbations are made to the spatial proximity of the cells within the population.

At step 140, the genetic status data captured at step 130 is subject to further analysis. In particular, the mutation status of an ancestor cell and its progeny cells at different cell cycle generations are identified and compared to extrapolate lineage and phylogenetic information and/or cellular event history.

In one aspect, the method and system disclosed herein are capable of capturing or recording multiple molecular changes over time; it is not limited to registering a single change.

To this end, in some embodiments, multiple “scratchpads” are specified in the cell genome. A genetic scratchpad can be any polynucleotide sequence whose sequence information is at least partially known. A scratchpad can be “written on” and serves as a unique recording or capturing site.

Scratchpads can be synthetic and composed of a variety of elements including repetitive segments, homology regions flanking a central core comprising the repetitive segments and one or more promoter sequences, and enzymatic recognition sequences. Scratchpad units may be a range of lengths and include various upstream promoters or other elements and different downstream sequences. They can be introduced into the genome as separate units or as part of a larger integrated cassette, like an artificial chromosome. Alternatively, scratchpads can also utilize the endogenous genomic DNA and not require synthetic additions.

In some embodiments, a genetic scratchpad comprises nucleotide sequences that are synthesized in vitro and then introduced into cells by methods such as transfection.

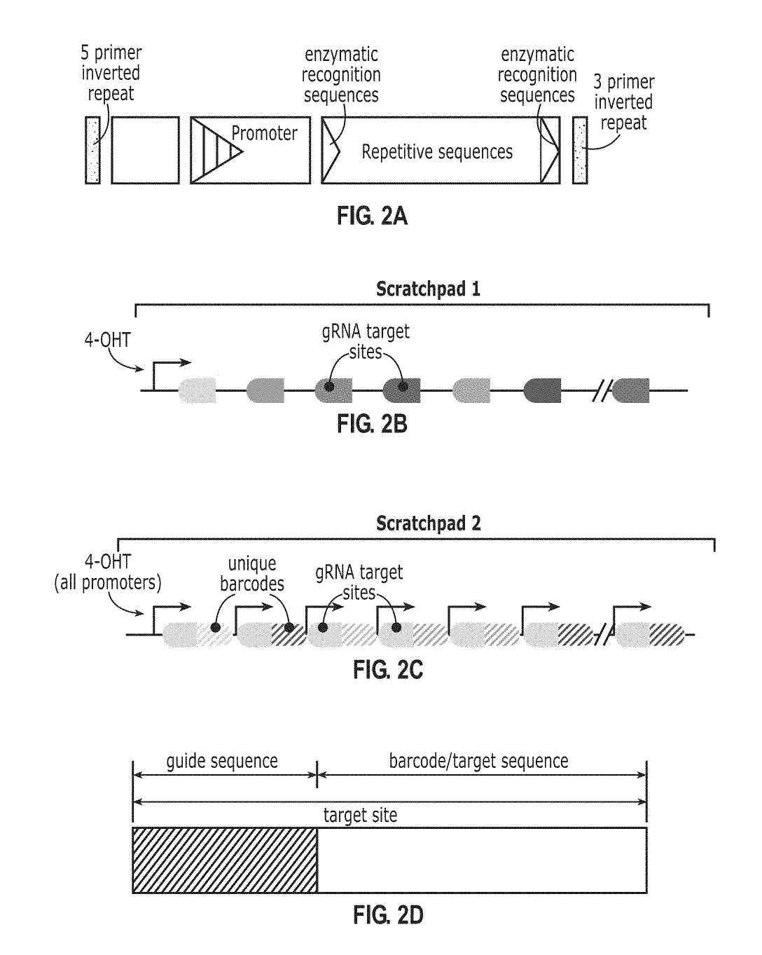

FIG. 2A depicts an exemplary embodiment, illustrating the basic scratchpad configuration, from left to right, which includes a 5 prime inverted repeat for integration (thin rectangle), an insulated promoter region (rectangular box with an arrow), a repetitive region flanked by enzymatic recognition sequences (thin arrowheads), and 3 prime inverted repeat (thin rectangle).

In some embodiments, an implementation of this strategy involves a scratchpad with a repetitive sequence at its core that can be deleted (FIG. 2A); for example, by enzyme that can recognize the recognition sequences that flank the repetitive sequences. In some embodiments, the scratchpad has multiple target sites and the repetitive sequences are inserted at different target sites in the scratchpad. In some embodiments, such repetitive sequences are inserted into multiple scratchpads.

In some embodiments, an implementation of this strategy involves a scratchpad with a repetitive sequence at its core that can be deleted (FIG. 2A). In such embodiments, a genetic scratchpad comprises one or more target sites with such a repetitive sequence. In some embodiments, these target sites comprise different number of copies of such repetitive sequences. For example, scratchpad A has 5 target sites. Target site 1 has 3 copies of the repetitive sequences while target site 2 can have 5 or more copies of the same repetitive sequences and etc. Because the repetitive sequences are between enzyme cleavage sites, by altering the number of repetitive sequences, different target sites can be identified by using methods that can assess the length of the resulting genetic scratchpad. An exemplary method includes single cell based polymerase chain reaction (PCR) analysis.

In some embodiments, though the core of the scratchpad is the same in each case, the sites can actually be differentiated because they are flanked by distinct genomic regions. The genomic context of each scratchpad can be identified individually by PCR and/or next generation sequencing methods, providing a unique target sequence or “barcode” for each scratchpad. For example, one characterized line has at least 10 scratchpads spread across unique genomic regions on 7 chromosomes. Unique target sequence or barcodes can also be created by other means, including constructing scratchpads with different unique synthetic sequences.

In some embodiments, multiple copies of this scratchpad can be introduced throughout the genome by transposase mediated recognition of inverted repeats (FIG. 2A), or other means, creating a large number of unique target sites. Molecular changes at these target sites will be captured or recorded.

In some embodiments, the scratchpad can contain other features, such as a promoter that allows transcription of this scratchpad and helps with readout (a feature described further below).

In alternative embodiments, a genetic scratchpad is located in defined regions within the natural genome of a cell. Because the sequence information of the genome of many organisms, including humans, is known, a genetic scratchpad can be defined based on the sequence information of selected genetic regions of interest in a genome. For example, sequences near or at genetic regions of interest (e.g., a target site) can be designated as a guide sequence to recruit one or more secondary molecules (e.g., a guide RNA known as a gRNA and a nuclease that is recruited by the gRNA), which facilitate the occurrence of certain molecular changes at the genetic regions of interest. In some embodiments, a nick or a double stranded break is created by the one or more secondary molecules resulting in disruption of the genetic region of interest, which can then be detected by the characterization component.

In still alternative embodiments, synthetic guide sequences can be inserted into selected regions within the natural genome of a cell. In some embodiments, such guide sequences are located at or near regions of interest such as target sites. As disclosed herein above, the guide sequences can recruit one or more secondary molecules (e.g., a guide RNA known as a gRNA and a nuclease that is recruited by the gRNA), which facilitate the occurrence of certain molecular changes at the genetic region of interest.

As disclosed herein, a cell can have one or more genetic scratchpads. In some embodiments, a cell has two or more genetic scratchpads, such as between three and five genetic scratchpads. In some embodiments, a cell has five or more genetic scratchpads, such as between five and nine genetic scratchpads. In some embodiments, a cell has 10 or more genetic scratchpads, such as between 10 and 15 genetic scratchpads. In some embodiments, a cell has 15 or more genetic scratchpads, such as between 15 and 19 genetic scratchpads. In some embodiments, a cell has 20 or more genetic scratchpads, 25 or more genetic scratchpads, 30 or more genetic scratchpads, 40 or more genetic scratchpads, 50 or more genetic scratchpads, 60 or more genetic scratchpads, 70 or more genetic scratchpads, 80 or more genetic scratchpads, 90 or more genetic scratchpads, 100 or more genetic scratchpads, 120 or more genetic scratchpads, 150 or more genetic scratchpads, 180 or more genetic scratchpads, 200 or more genetic scratchpads, or 500 or more genetic scratchpads.

In some embodiments, the number of genetic scratchpads in a particular genomic is determined by the complexity of the lineage information. For example, the number of genetic scratchpads required for assessing the lineage information cross 10 possible regions of interest will be larger than that required for assessing the lineage information cross 3 or 5 possible regions of interest.

In some embodiments, the entire sequence information of the genetic scratchpad is known. In some embodiments, only a part of the sequence information of the genetic scratchpad is known.

Also as disclosed, a genetic scratchpad comprises a polynucleotide sequence of any length. In some embodiments, the polynucleotide comprises 100 nucleotides or longer; 200 nucleotides or longer; 300 nucleotides or longer; 400 nucleotides or longer; 500 nucleotides or longer; 700 nucleotides or longer; 1,000 nucleotides or longer; 1,500 nucleotides or longer; 2,000 nucleotides or longer; 2,500 nucleotides or longer; 3,000 nucleotides or longer; 4,000 nucleotides or longer; 5,000 nucleotides or longer; 6,000 nucleotides or longer; 7,000 nucleotides or longer; 8,000 nucleotides or longer; 10,000 nucleotides or longer; 12,000 nucleotides or longer; 15,000 nucleotides or longer; 20,000 nucleotides or longer; 50,000 nucleotides or longer; or 100,000 nucleotides or longer.

Preliminary modeling suggests that, in order to allow proper tracking of lineage information, an ideal system would provide at least two mutations per generation per scratchpad. To track about 10 generations, about 100 target sites should be sufficient.

A genetic scratchpad comprises multiple target sites, as depicted in the exemplary genetic scratchpads in FIGS. 2B and 2C. In some embodiments, each target site comprises a binding site that is recognized by a guide molecule such as a guide RNA (gRNA). In some embodiments, each target site comprises a target sequence or barcode associated with a guide molecule binding site.

FIG. 2D illustrates an exemplary target site, for example, those corresponding to those depicted in FIG. 2C. In such embodiments, the target site comprises a guide sequence with a segment that is recognized by a gRNA. In some embodiments, the gRNA has a complementary sequence that allows the gRNA to bind to the guide sequence. In some embodiments, the sequence in the gRNA can be adjusted to modify the binding interactions between the gRNA and the guide sequence within a target site. Such adjustment is used to modulate the frequency at which the gRNA binds to the guide sequence and thereby modulating the frequency at which any molecular events that may occur upon binding between the gRNA and the guide sequence.

In some embodiments, when a gRNA binds to its corresponding guide sequence, it recruits one or more secondary molecules, which then trigger one or more molecular changes. For example, an enzyme such as Cas9 nuclease can be recruited to the gRNA binding site. The nuclease then creates nicks or double-stranded break at the binding site, thereby destroying the structural integrity of a target site.

In some embodiments, all or at least a part of the guide sequence is also recognized by a molecule that is used to characterize the integrity of a target site. For example, such a molecule can be a hybridization probe for fluorescence imaging analysis.

In some embodiments, a target site further comprises a barcode or target sequence. All or at least a part of the barcode or target sequence is also recognized by a molecule that is used to characterize the integrity of a target site. For example, such a molecule can be a hybridization probe for fluorescence imaging analysis.

In some embodiments, the length of the guide sequence is typically at least 20 nucleotides. However, guide sequences can be shorter or longer to modify their associated efficiency in recruiting secondary molecules. Additionally, to target multiple sequences, with a signal guide RNA molecule, guide sequences can be arranged in tandem with intervening spacer regions.

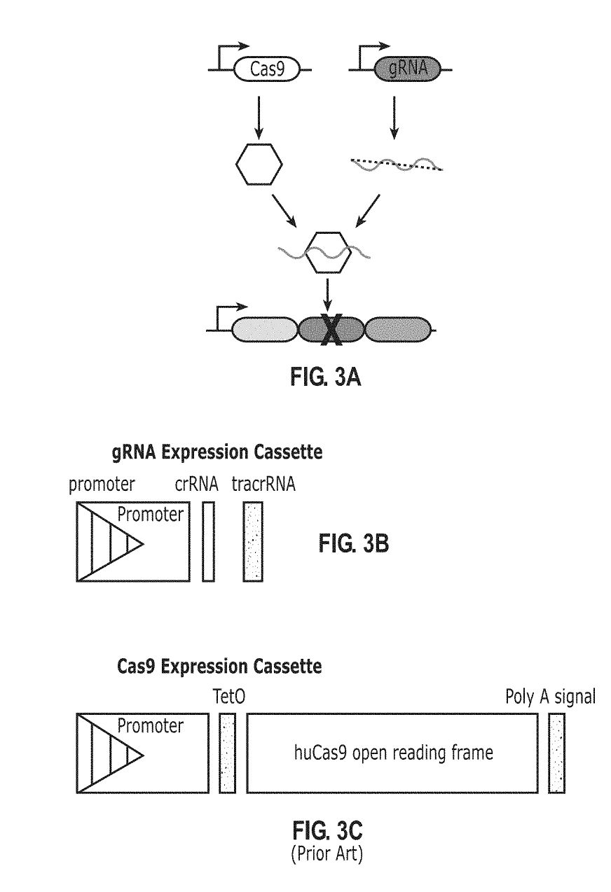

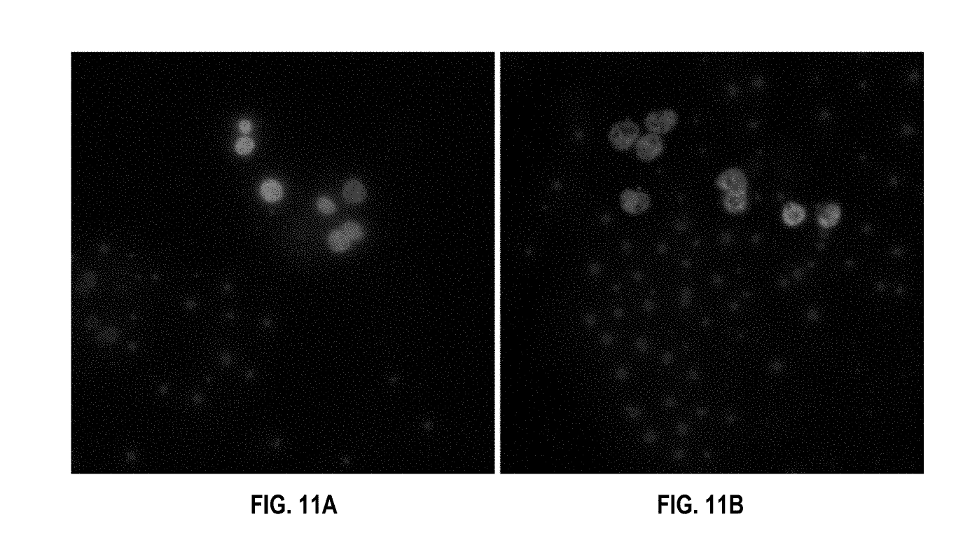

In some embodiments where multiple scratchpads are present in a genome, each scratchpad can be independently written (e.g., via enzymatic cleavage of repetitive sequences) or using a genomic editing tool such as the Clustered Regularly Interspaced Short Palindromic Repeats (CRISPR) system (e.g., through a guide RNA and the Cas9 nuclease) (FIGS. 3A-3C). Presence of Cas9 and a specific guide RNA (gRNA) in the system leads to deletion of the scratchpad core, a change readily detected in bulk (FIG. 3) and in situ (FIG. 11).

In one aspect, provided herein is a writing component that is capable of creating the molecular changes to be captured or recorded.

In order to capture or record the molecular changes, a writing component should trigger or create molecular changes only in defined regions, for example, within a target site. This way, changes brought about by the molecular changes can be assessed in subsequent characterization analysis. To this end, a writing component comprises a guide molecule. The main function of the guide molecule is to recognize a desired target site. In some embodiments, the guide molecule is an RNA molecule that associates itself to the desired target site via complementary sequence recognition. In some embodiments, other molecules may facilitate the recognition and association between the guide molecule and the desired target site.

In addition, the writing component comprises one or more secondary molecules that are capable of triggering or creating one or more molecular changes at the desired target site. In some embodiments, one or more secondary molecules are recruited by the guide molecule to the target site. In some embodiments, the guide molecule binds to a guide sequence first to form a complex, which is then recognized by one or more secondary molecules. In some embodiments, the guide molecule and one or more secondary molecules bind first before the complex recognizes and binds to the guide sequence at the target site.

In some embodiments, the Clustered Regularly Interspaced Short Palindromic Repeats (CRISPR) system, one of the most commonly used RNA-Guided Endonuclease technologies for genome engineering, can be used as a writing component. Exemplary embodiments of the CRISPR system are depicted in FIGS. 3A through 3C.

In a CRISPR system, the guide molecule is a gRNA (e.g., FIG. 3A). When the gRNA binds to a guide sequence in the target site, it recruits secondary molecules (e.g., Cas9 nuclease) to trigger subsequent molecular changes: nicks or break in nucleotide sequences, which leads to various genetic mutations. Such genetic mutations include but are not limited to insertion mutation, deletion mutation, point mutations, multiple point mutations, any combination of such mutations, or any other changes at the nucleic acid level that can affect the binding of guide molecules such as gRNAs. Insertion and deletion mutations (also referred to as indel mutations) often lead to frame shift mutations leading to major disruptions in one or more genes, as illustrated in FIG. 3A. As such, probes designed to recognize the original target site will no longer be able to bind to the disrupted region. Alternatively, molecular changes include genetic modification. For example, a methyl-transferase can be fused to cas9 and target specific sites to alter the subsequent activity of a target site in one or more genetic scratchpads. Methylation on the DNA can be detected by bi-sulfite conversion, which turns unmethylated Cs to Us.

A typical CRISPR system comprises two independent cassettes for expressing its two distinct components: (1) a guide RNA and (2) an endonuclease such as the CRISPR associated (Cas) nuclease, Cas9.

The guide RNA is a combination of the endogenous bacterial crRNA and tracrRNA into a single chimeric guide RNA (gRNA) transcript. The gRNA combines the targeting specificity of the crRNA with the scaffolding properties of the tracrRNA into a single transcript. An exemplary gRNA expression cassette (e.g., FIG. 3B) depicts an RNA polymerase III or polymerase II specific promoter (box with an arrowhead), which drives the expression of a chimeric crRNA (middle rectangle) and tracrRNA (far right, shaded rectangle).

An exemplary Cas9 expression cassette is found in FIG. 3C, which shows an RNA polymerase II promoter (rectangle with an arrowhead), an array of two binding sites for a repressor protein (TetR) and a “humanized” huCas9 open reading frame followed by poly A signal from the bovine growth hormone gene (dark, shaded rectangle). When the gRNA and the Cas9 nuclease are expressed in the cell, the genomic target sequence can be modified or permanently disrupted.

The gRNA/Cas9 complex is recruited to the target sequence by the base-pairing between the gRNA sequence and the complement to the target sequence in the genomic DNA. In some embodiments, to ensure successful binding of Cas9, the genomic target sequence also contains the correct protospacer adjacent motif (PAM) sequence immediately following the target sequence. The binding of the gRNA/Cas9 complex localizes the Cas9 to the genomic target sequence so that the wild-type Cas9 can cut both strands of DNA causing a double strand break (DSB). Cas9 cuts 3-4 nucleotides upstream of the PAM sequence.

Recent publication and preliminary experiments suggest that Cas9 can be a suitable component for “writing” random mutations into an engineered scratchpad region in the genome, where the scratchpad comprises many individually addressable target sites for the gRNA-Cas9 complex (FIGS. 2B and 2C). Aspects of the Cas9 system enable tuning of the rate of mutagenesis and scaling of the size of the target region.

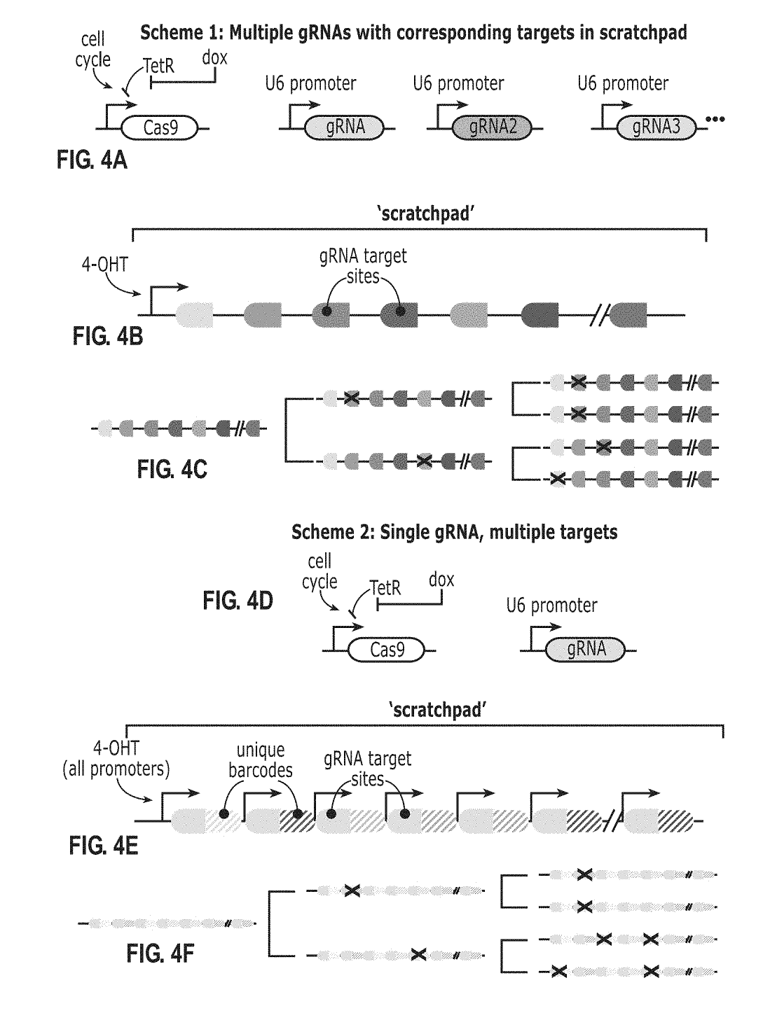

FIGS. 4A through 4F illustrate two exemplary schemes for creating genetic mutations into genetic scratchpads. In each one, a set of expression constructs (FIGS. 4A and 4D), a corresponding scratchpad (FIGS. 4B and 4E) and a schematic 3-generation lineage tree (FIGS. 4C and 4F) are shown. X's indicate mutations.

In Scheme 1, the CRISPR system includes one Cas9 protein but multiple gRNAs (e.g., FIG. 4A). In some embodiments, the gRNAs are all under the control of a U6 promoter. Each gRNA binds to a unique target site in a genetic scratchpad and subsequently recruits the Cas9 nuclease to create a mutation at the target site (e.g., FIG. 4B). The site of the mutations may depend on the binding efficiency of the particular gRNA or the cutting efficiency of the Cas9 nuclease at the site.

In some embodiments, multiple mutations accumulate over multiple cell cycle generations. For example, as illustrated in FIG. 4C, the genetic scratchpad of FIG. 4B leads to two possible mutations in its first generation offspring: one comprising a mutation at target site No. 2 and the other comprising a mutation at target site No. 5. The mutations are preserved in the offspring of these two first generation offspring.

In some embodiments, additional mutations are created in addition to those carried over from the parent generation. In some embodiments, no additional mutations are created in one or more generations. For example, as depicted in FIG. 4C, in the next generation, no additional mutation is introduced into the scratchpad containing the mutation at target site No. 2. However, the scratchpad carrying the mutation at target site No. 5 leads to two offspring with double mutations: one with mutations at target site No. 3 and site No. 5 and the other at target site No. 1 and No. 5.

In some embodiments, it is also possible for multiple mutations to occur in subsequent generations, such as two or more mutations, three or more mutations, or even five or more mutations. In order to keep the number of mutations under a reasonable limit and better assess lineage information between different generations, various methods (e.g., by applying mismatching sequences in a gRNA to adjust the rate at which it binds to a guide sequence) are applied to adjust the occurrence rate of mutations.

In Scheme 2, only a single gRNA is used against multiple target sites (e.g., FIG. 4D). Here, instead of having unique gRNAs bind to different target site, each target site includes a unique barcode or target sequence to which unique probes can bind to reveal the presence of a particular target site (e.g., FIG. 4E). The detailed recognition mechanism will be described in the following section.

Similar to the setup of Scheme 1, binding of the gRNA to a target site also ultimately leads to mutations after a Cas9 nuclease is recruited. Also similarly, such mutations can be preserved in future generations. Further, additional mutations can occur at different target sites in future generations of cells.

As illustrated, lineage trees can be inferred from determination of the patterns of mutations (e.g., FIGS. 4C and 4F).

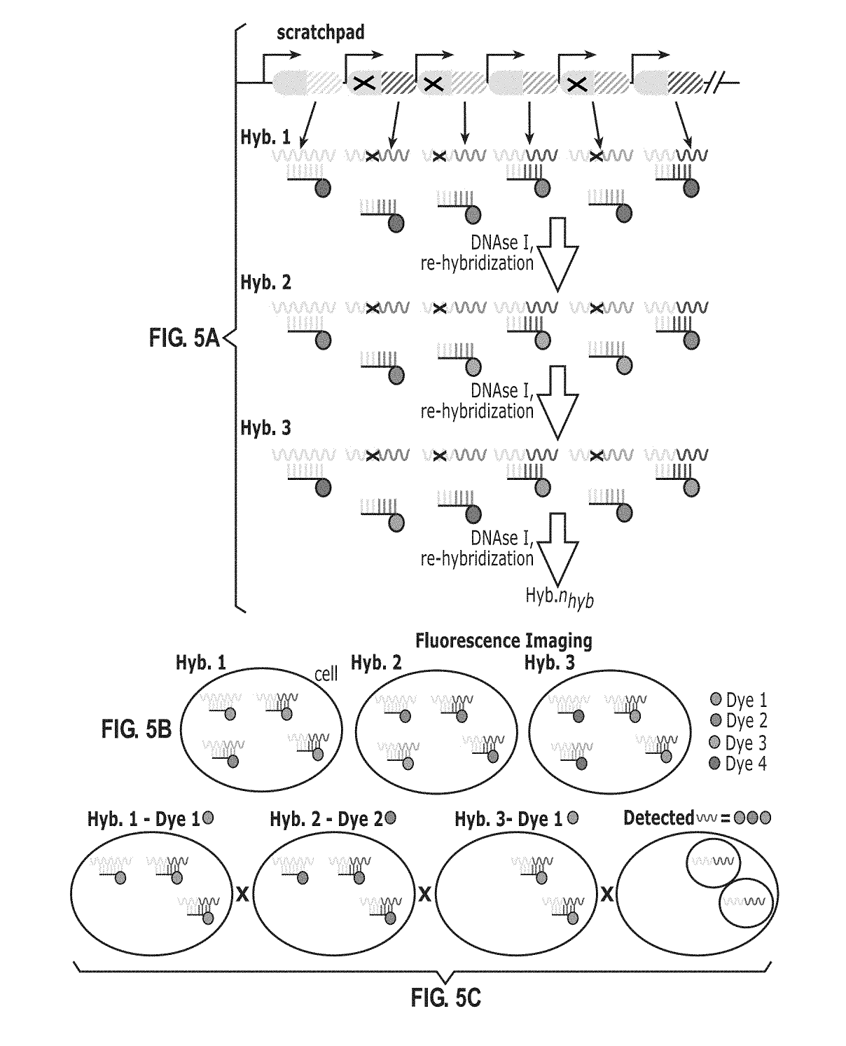

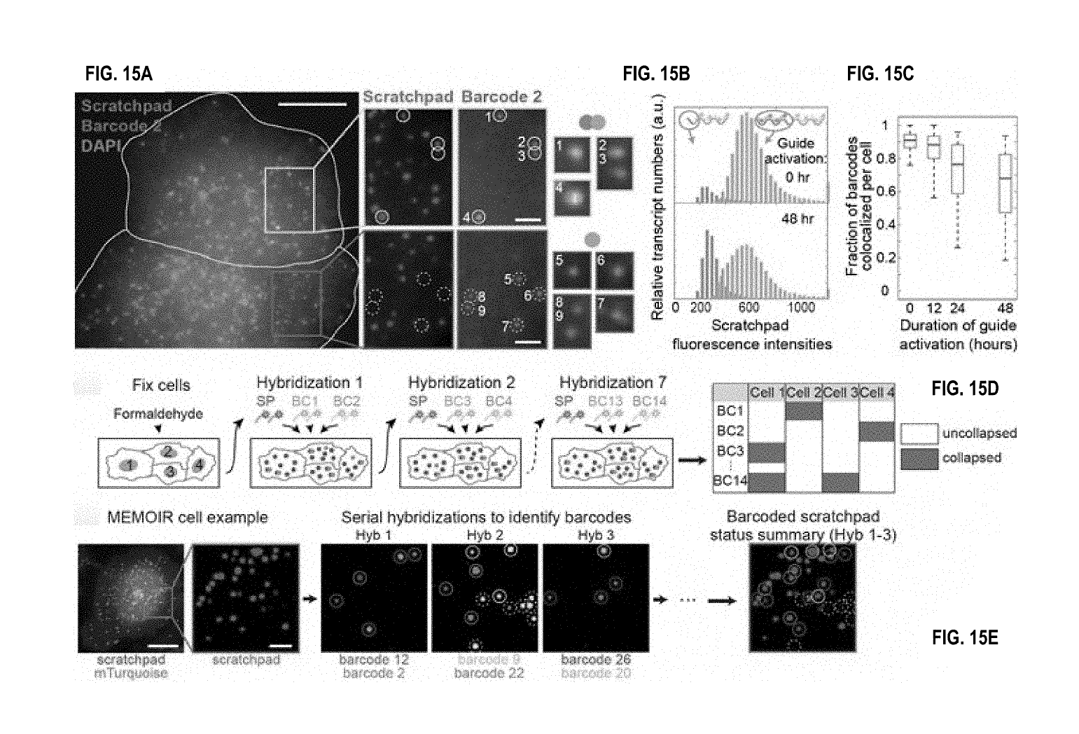

Scheme 1 is optimized for single-cell DNA sequencing detection of mutations, while Scheme 2 is optimized for detection by multiplexed smFISH (e.g., FIG. 5). In both schemes, the scratchpads can be transcribed from a promoter. The promoter can be either inducible or constitutive. Expression enables mutations to be read out by hybridization to RNA (FIG. 5). Actual experimental data corresponding to the schematic representation in FIG. 5 can be found in FIG. 15.

In one aspect, provided herein are methods and systems for characterizing the location of mutations in one or more genetic scratchpads.

In some embodiments, single-cell sequencing techniques can be used to reveal the mutations in the target sites in one or more scratchpads before standard computational methods are applied to determine lineage relationships.

In some embodiments, to readout the mutations made on the scratchpad in situ, a recently developed method is adapted to identify mutations in single cells within complex tissues while preserving spatial information. In some embodiments, the expression of the recording region into RNA is induced from an upstream inducible promoter (e.g., FIGS. 4A and 4D). This has two benefits. First, it allows the application of single molecule fluorescent in situ hybridization (smFISH), which is already optimized for RNA detection. As disclosed herein, smFISH can be used interchangeably with FISH unless otherwise specified. In addition, transcription amplifies the signal, as multiple copies of each mRNA are expressed from the scratchpad region, which enhances detection efficiency and accuracy.

To uniquely distinguish the different target sites on the scratchpad, unique barcode sequences are engineered at each target site (FIG. 4E). smFISH probes recognizing such unique sequence are designed to span the junction across the target site and the barcoded region, and are thus sensitive to mutations in or near the target. In some embodiments, these mutations are large insertions or deletions, which are readily detected by smFISH probe hybridization.

In some embodiments, it is possible to detect indels or minor mutations such as single point mutations and multiple point mutations. Recent work has shown that single nucleotide polymorphisms (SNPs) on individual transcripts can be efficiently detected by 25mer smFISH probes.

As disclosed herein, indel mutations are suitable molecular changes for a couple of reasons. First, indels are easier to detect than SNPs, since frameshifts are more disruptive to hybridization than mutations. Second, as the RNA is overexpressed from the reading template region, a large number of transcript copies can be analyzed in each cell, boosting the detectable signal.

In some embodiments, probes used to recognize and bind to an mRNA transcript or a DNA sequence are oligonucleotides, or oligos. In some embodiments, the oligo probes are 10-mer or shorter. In some embodiments, the oligo probes are 15-mer or shorter. In some embodiments, the oligos are 20-mer or shorter; 25-mer or shorter; 30-mer or shorter; 40-mer or shorter; 50-mer or shorter; 70-mer or shorter; 100-mer or shorter; 150-mer or shorter; 200-mer or shorter; 250-mer or shorter; 300-mer or shorter; 500-mer or shorter; or 1,000-mer or shorter.

In some embodiments, the oligo probes are designed by using complementary sequences to randomly selected sequences or segment of sequences in a target sequence (e.g., an mRNA or DNA sequence).

In some embodiments, the oligo probes are designed by deliberately selecting sequences or segments of sequences that bind to a target site (e.g., an mRNA or DNA sequence) with known or predicted binding affinity. This is called “intelligent probe design,” where structure, sequence and biochemical data are all considered to create probes that will likely have better binding properties to a target site. In particular, the preferred regions to be used as target sites in a genome are either identified experimentally or predicted by algorithms based on experimental data or computation data. For example, computed binding energy and/or theoretical melting temperature can be used as selection criteria in intelligent probe design.

Tools are available for automated designs of probes that will have either actual or predicted optimal binding properties to the target site. For example, the Designer program is routinely used for designing probes that bind to a particular target RNA sequence as part of the established single molecule RNA Fluorescent in-situ hybridization technology (smFISH), which was developed at the University of Medicine and Dentistry of New Jersey (UMDNJ) a Single Molecule Fluorescent in-situ hybridization technology based on detection of RNA (singlemoleculefish<dot>com/designer<dot>html). For the Designer program, the open reading frame (ORF) of the gene of interest is typically used as input. This approach is used to exclude the more repetitive regions and low complexity sequence contained in Un-translated Regions (UTRs). Probes are designed to minimize deviations from the specified target GC percentage. The program will output the maximum number of probes possible up to the number specified. Sequence input is stripped of all non-sequence characters. A user can specify parameters such as the number of probes, target GC content, length of oligonucleotide and spacing length. Most success has been achieved with target GC contents of 45%. Typically, oligos are designed as 20 nucleotides in length and are spaced a minimum of two nucleotides apart.

One of skill in the art would also understand that length or size of probes will vary, depending on the target sites, genetic scratchpad and purposes of the analysis.

Additional description on single molecule FISH can be found in, for example, Raj A., et al., 2008, “Imaging individual mRNA molecules using multiple singly labeled probes,” Nature Methods 5(10): 877-879; Femino A., et al., 1998, “Visualization of single RNA transcripts in situ,” Science 280: 585-590; Vargas D., et al., 2005, “Mechanism of mRNA transport in the nucleus,” Proc. Natl. Acad. Sci. of USA 102: 17008-17013; Raj A., et al., 2006, “Stochastic mRNA synthesis in mammalian cells,” PLoS Biology 4(10):e309; Maamar H., et al., 2007, “Noise in gene expression determines cell fate in B. subtilis,” Science, 317: 526-529; and Raj A., et al., 2010 “Variability in gene expression underlies incomplete penetrance,” Nature 463:913; each of which is hereby incorporated by reference herein in its entirety.

Any suitable labels can be associated with the specific probes to allow them to emit signals that will be used in subsequence imaging analysis. In some embodiments, the same type of labels can be attached to different probes for different target sites.

One of skill in the art would understand that choices for a label are determined based on a variety of factors, including, for example, size, types of signals generated, manners attached to or incorporated into a probe, properties of the target sites including their locations within the cell, properties of the cells, types of interactions being analyzed, and etc.

In some embodiments, all the target sites on the scratchpad are scanned to determine the target sites that are mutated in each cell. In some embodiments, a method to multiplex mRNA detection in single cells in situ is applied. In this approach, the mRNAs in cells are barcoded by sequential rounds of hybridization, imaging, and probe stripping (FIGS. 5A through 5C). As the transcripts are fixed in cells, the fluorescent spots corresponding to single mRNAs remain in place during multiple rounds of hybridization, and can be aligned to read out a color sequence at each point in the cell. This temporal barcode is designed to uniquely identify an mRNA species in a multiplexed experiment. During each round of hybridization, each transcript is targeted by smFISH probes labeled with one dye. The sample is imaged and treated to remove the smFISH probes. Then the mRNA is hybridized in a subsequent round with the same smFISH probes labeled with a different dye. The number of barcodes available with this approach scales as F^{N}, where F is the number of fluorophores and N is the number of hybridization rounds. For example, with 4 dyes, 8 rounds of hybridization can cover the entire transcriptome (4⁸=65,536).

Using smFISH and fluorescent microscopy to analyze mutation events has the significant advantage compared to DNA-seq that single cells do not need to be extracted from tissues. Spatial context is preserved. For example, it is possible with this approach to visualize individual cells within a brain slice to determine the mutation set in each of those cells. This not only preserves the spatial information, but is less labor and cost intensive to perform. With conventional fluorescent microscopy, a 1 mm×1 mm×1 mm region can be scanned in approximately 5 minutes. The entire mouse brain can be imaged in 100 hours. With an automated microscope, 4 rounds of hybridization can be performed in 2-3 weeks. The overall cost of the microscope time and reagents will be approximately $10-50 k per brain. In comparison, single cell DNA sequencing costs approximately $10 per cell at the present, and dissecting out more than 1000 cells would be prohibitively labor intensive and cost prohibitive. Lastly, it is possible to apply this approach to CLARITY cleared brains to obtain lineage information directly from intact brains.

FIGS. 5A through 5C depict an exemplary process for detecting mutations in a genetic scratchpad by RNA hybridization smFISH. FISH probes used here include sequence that binds to all or a part of guide sequence and all or a part of the barcode or target sequence adjacent or near the guide sequence. Fluorescent signals are only emitted when the smFISH probes bind to un-mutated sequences. Disruption of either sequence will lead to loss of signal.

As disclosed previous, disruption by Cas9 results in mutations in the guide sequence (e.g., insertion, deletion or point mutations). Such mutations, in particular, the insertion and deletion mutations prevent a smFISH probe from binding to both the guide sequence and/or barcode sequence.

Here, scratchpads are expressed as mRNAs to enable detection of mutations using FISH probes in individual cells. Using sequential rounds of hybridization (Hybs. 1, 2, 3, . . . ) multiple target sites can be probed simultaneously in single cells. In each round of hybridization, a mutation is targeted by a smFISH probe with the same sequence but a different dye (e.g., FIG. 5A). Thus, each mutation can be addressed by a particular dye sequence.

For example, the genetic scratchpad here contains 3 mutations, at target sites No. 2, No. 3 and No. 5. In three rounds of hybridization, probes recognizing different target sites are as follows.

| | | Probe Color | Probe Color | Probe Color |
|---|---|---|---|---|
| | Mutation? | (Round 1) | (Round 2) | (Round 3) |
| Target site No. 1 | No | Blue | Green | Red |
| Target site No. 2 | Yes | Blue | Green | Orange |
| Target site No. 3 | Yes | Green | Orange | Red |
| Target site No. 4 | No | Green | Orange | Blue |
| Target site No. 5 | Yes | Red | Orange | Green |
| Target site No. 6 | No | Blue | Green | Blue |

After the mutations, only intact target sites are able to produce fluorescent signals. Sequential hybridizations determine which transcripts are both present and do not contain mutations.

At each hybridization step, cells are imaged in all channels. Color dots in cells correspond to probes hybridizing to indicated transcripts (FIG. 5B). Each round of hybridization results in a snapshot of the cell containing multiple fluorescent signals. Here, it is possible to detect the signal from the same target site multiple times, because multiple copies of mRNA can be synthesized.

Because the characterization is done in situ without disrupting the structural integrity of the cells, it is possible to observe multiple color sequences for the same target site after each round of hybridization. The order by which the color signals appear forms a unique code for identifying the particular target site.

By multiplying or, more generally, cross-correlating images in different rounds of hybridization, one can specifically detect the color sequence of any desired transcript. For example, here the intact target site No. 6 is uniquely detected by combining the blue Hyb 1 image with the green Hyb 2 image and the blue Hyb 3 image (FIG. 5C).

As listed in the table above, by alternating the colors of different probes and applying multiple round of hybridization, each target site corresponds to a particular color sequence code. Here, intact site No. 1 will produce blue, green, and red signals in the order specified. Intact site No. 4 will produce red, orange, and green signals in the order specified. Intact site No. 6 will produce blue, green, and blue signals in the order specified.

One of skill in the art would understand that, when more target sites are involved, more rounds of hybridization will be performed to establish color code sequences that can sufficiently and uniquely identify any intact target site

In some embodiments, other in situ readout methods can also be applied to characterize the mutation status of target sites with one or more genetic scratchpads. Beyond RNA FISH, it is possible to use DNA FISH for in situ readout of recorded events. Expression changes to fluorescence reporters could also be used (in both live and fixed cells), though limits on the number of distinct fluorophore colors could cap the number of recordable events. Other readout methods could also provide in situ-like information, such as single-cell sequencing or PCR when implemented to preserve spatial information. Further, multiple techniques (including single-cell sequencing and PCR) could be readily applied to verify population averages.

Methods and systems described herein enable the reconstruction of lineage trees based on the historical record of induced mutations recorded in scratchpads. More importantly, the recorded information can include data on specific molecular events that occurred in each branch of the tree over time. Exemplary events include but are not limited to activation of master transcription factors or signaling pathways.

To achieve event recording, provided herein are strategies for simultaneously recording lineage information and molecular events.

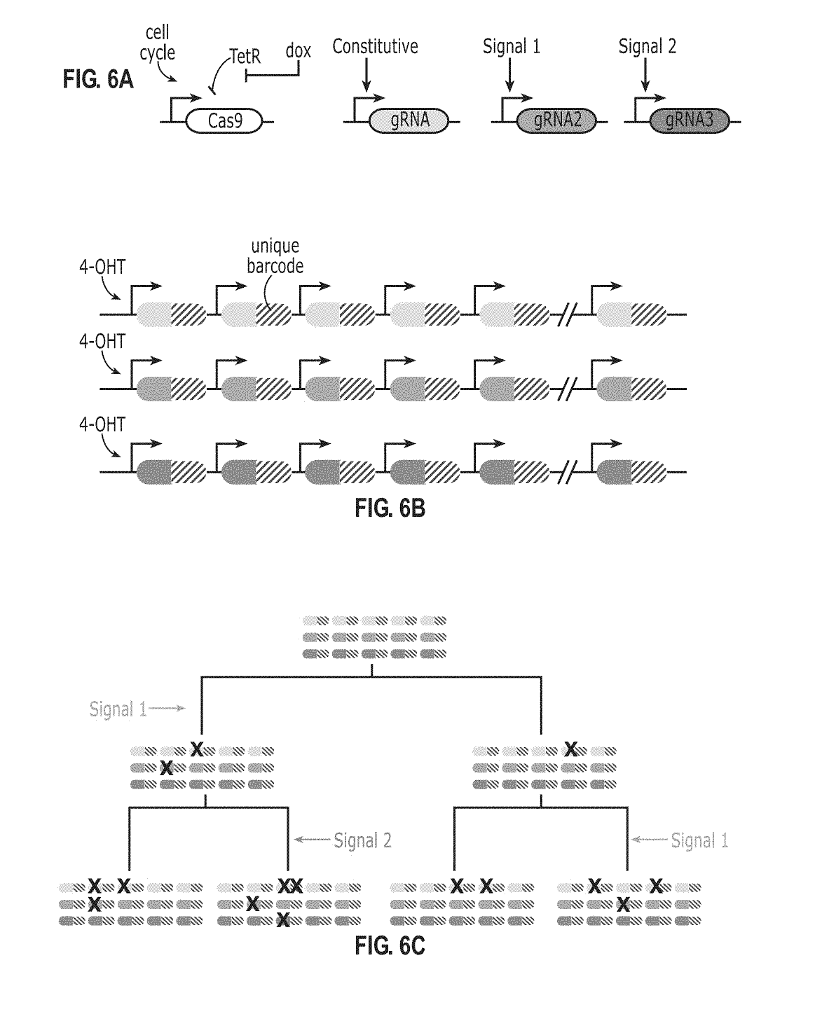

In some embodiments, constitutive and conditional focused mutagenesis systems are coupled. In an exemplary embodiment, a set of gRNAs is activated by a particular constitutive promoter, and is identical with the system discussed previously in connection with event writing. Each additional set will be conditional, being activated by a transcription factor of interest. It will consist of a promoter sensitive to that transcription factor driving a distinct gRNA, which will in turn target a distinct set of barcoded spacers in scratchpad target sites. Reading out of genotypes, as previously described, will be extended to include the additional scratchpads regions. The key idea is that the conditional systems will generate mutations only during intervals when the corresponding gRNA is expressed. By superimposing mutagenic events from the constitutive and signal-dependent gRNAs, one can reconstruct not just the lineage tree, but also the branches in which signaling events occurred (e.g., FIG. 6).

In the exemplary embodiment depicted in FIG. 6, multiple focused mutagenesis systems are used, each of which utilizes a distinct set of gRNAs and corresponds to a genetic scratchpad.

FIGS. 6A through 6C illustrate that event recording can be integrated into the lineage tracking system using an intersectional strategy. FIG. 6A depicts an exemplary design of one potential event recording system. Cas9 is expressed from a cell cycle dependent promoter and a constitutive promoter drives one guide RNA (gRNA1), as above. In addition, two signal-dependent promoters drive distinct gRNAs (e.g., gRNA2 and gRNA3) that target additional corresponding scratchpads (e.g., FIG. 6B). As a result, signaling events that occur during development can be recorded alongside lineage information, as indicated schematically by the mutations (X's) in (FIG. 6C). While mutations associated with the constitutive promoter can occur during any cell cycle, the mutations controlled by signal-dependent promoters can be turned on and off. This way, certain mutations (e.g., those associated with gRNA2 and gRNA3) are induced only in specific cell cycle.

Signaling pathways provide a model system for recording known inputs. In some embodiments, signaling pathways such as BMP, SHH, and Notch will be analyzed by the methods and systems disclosed herein. Such pathways are critical for diverse developmental processes, easy to manipulate with external ligands and pharmacological inhibitors, and in active use in the lab.

In some embodiments, these pathways will be activated or inhibited in mouse embryonic stem cells (mESCs) containing corresponding recording systems utilizing pathway specific sensors incorporating multimerized binding sites for Smad and CSL transcription factors, respectively.

Focused mutagenesis can enable “analog” recording of event intensity. Stronger signaling events are expected to induce higher expression of corresponding gRNAs, which could increase the mutation rate. As a result, the number of mutations accumulated in any given cell cycle could provide an indication not just of whether a transcription factor was active, but also of how strongly activated it was. To work, the mutation rate and number of target sites must be tuned to the dynamic range of the signal-dependent gRNA promoters. To explore this possibility, the relationship between ligand level and number of mutations induced will be systematically measured using the above signal pathways.

The event recording methods and systems disclosed herein can be used to analyze ES differentiation. In some embodiments, the methods and systems can be used to record the activation of master transcription factors that activate specific lineages under conditions of heterogeneous differentiation. In some embodiments, facts determined from gene expression (antibody staining or single-molecule RNA FISH) are correlated with records of transcription factor activation recorded in the scratchpad of the same cell.

As illustrated, the mutation status can be characterized in mammalian cells as well as simpler eukaryotic or even prokaryotic cells. In some embodiments, individual images of a cell population of interest are collected at different time points over a period of time. In some embodiments, continuous video images are collected over a period of time. In some embodiments, the period of time for image collection can cover any duration of time; for example, it can be over two cell cycle generations or longer, three cell cycle generations or longer, four cell cycle generations or longer, five cell cycle generations or longer, six cell cycle generations or longer, seven cell cycle generations or longer, eight cell cycle generations or longer, nine cell cycle generations or longer, 10 cell cycle generations or longer, 12 cell cycle generations or longer, 15 cell cycle generations or longer, 20 cell cycle generations or longer, 30 cell cycle generations or longer, 40 cell cycle generations or longer, 50 cell cycle generations or longer, 75 cell cycle generations or longer, or 100 cell cycle generations or longer.

In one aspect, provided herein are methods and systems for establishing or reconstructing lineage tree for a cellular process or pathway.

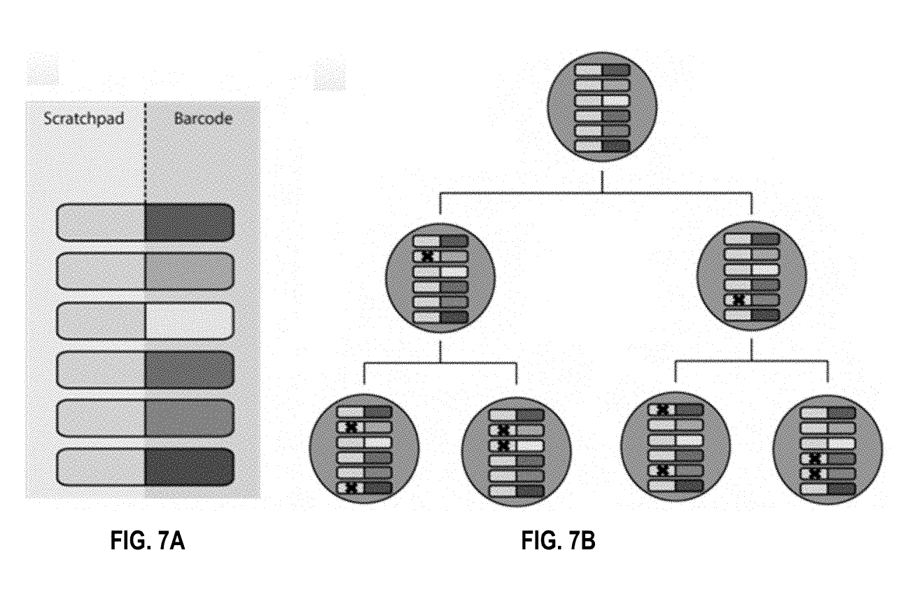

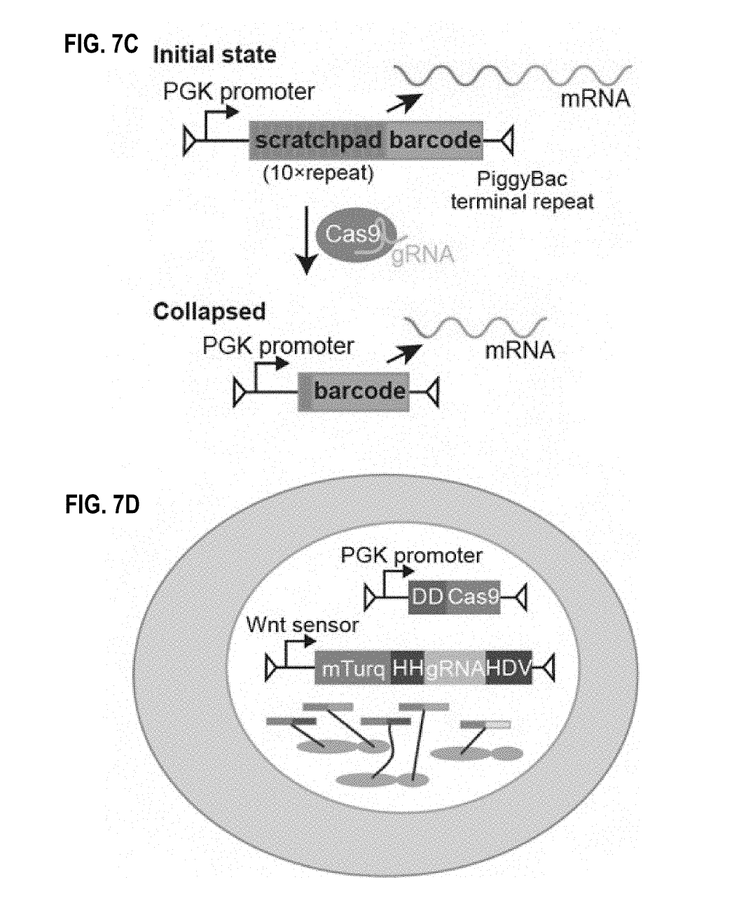

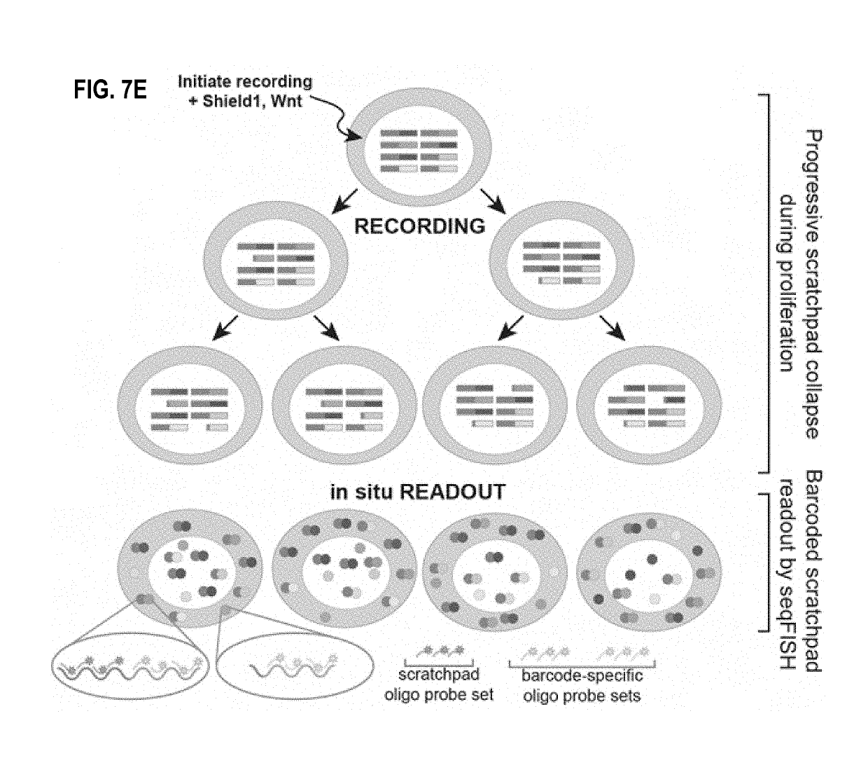

FIGS. 7A and 6E illustrate an exemplary schematic of lineage tree reconstruction based on scratchpad state. FIG. 6D depicts a scratchpad implementation including a region targeted for deletion (colored in gray in the left) and a unique barcode (in rainbow color on the right). FIG. 6D shows a lineage tree that is constructed based on deletions in the scratchpad (labeled as “x” in the figures). In particular, cells with common ancestors can be identified to reconstruct a lineage tree.

The method yields single-cell information and is not restricted to coarse-grained population measurements. It can also provide single-cell-cycle resolution: by adjusting the rate of scratchpad mutation, the time resolution of the technique can be tuned. In particular, mutation rates resulting in at least a few scratchpad mutations per cell cycle enable the reconstruction of lineage trees with single-cell resolution.

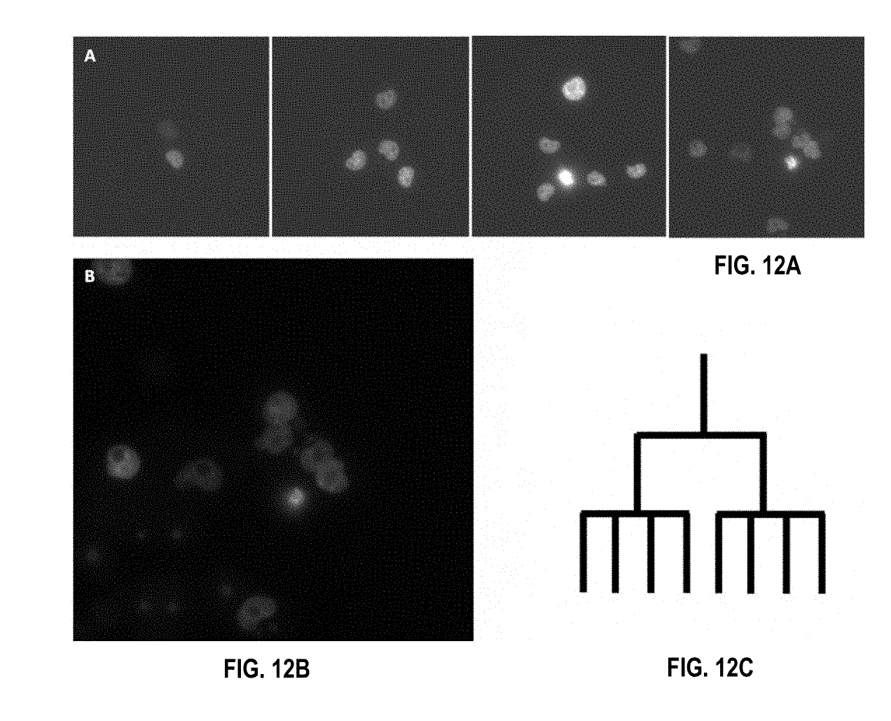

For example, lineage trees can be reconstructed based on inherited changes in each cell's scratchpad state. By reading out the accumulated changes in each cell, we can infer the most likely lineage history of a population of cells (FIGS. 7 and 12). Genomic changes induced by our method are deliberately tuned to occur more frequently than somatic mutations and are in defined locations, which provide improved lineage information (at single-cell resolution) and easier readout, respectively. Moreover, methods relying on somatic mutations are not currently amenable to in situ readout of the lineage information.

FIGS. 7C-7D illustrates an overall system for recording and in situ readout of cell lineage. For example, FIG. 7C shows a barcoded scratchpad that provides a general purpose recording element whose state can be irreversibly altered by Cas9/gRNA-mediated cleavage. Here, the promoter sequence and PiggyBac terminal repeats are specified in comparison to the more generic representation in, for example, FIGS. 2-4. FIG. 7D illustrates a recording system consists of three types of components, all stably integrated into the genome: (1) a Cas9 variant containing an inducible degron (DD) that is stabilized by the small molecule Shield1. (2) A Wnt-inducible gRNA targeting the scratchpad, co-expressed with a fluorescent protein (mTurquoise). Ribozyme sequences (HH, HDV) enable gRNA excision. (3) A set of barcoded scratchpads (two-colour elements) integrated throughout the genome. Inverted triangles in 7C and 7D denote PiggyBac terminal repeats, used for genome integration. FIG. 7E furthers illustrates an exemplary recording and readout process. During recording, scratchpads collapse stochastically as cells proliferate, producing distinct scratchpad states in each cell. During readout, individual mRNA molecules are detected with a single scratchpad-specific probe set (orange, inset), and multiple barcode-specific probe sets (blue, green, inset) through sequential rounds of hybridization and imaging. Uncollapsed scratchpads produce co-localized barcode and scratchpad signals (overlapping dots), while collapsed scratchpads produce only a barcode-specific signal (single dots).

FIG. 7 illustrates a system that corresponds to those illustrated FIGS. 2 through 6. In particular, FIG. 7C corresponds to FIGS. 2 and 3 where FIG. 2 illustrates the structure arrangements of a couple of genetic scratchpads and FIG. 3 illustrates how a Cas 9 and gRNA based system is used to delete sequence within the genetic scratchpad to result in a cut or collapsed genetic scratch pad.

More specifically, FIG. 3A illustrates a mechanism for mutating the scratchpad using CRISPR, which is the implementation actually used. FIG. 7C illustrates the actual mechanism of scratchpad mutation used in the paper: Cas9/gRNA target the scratchpad and cause it to collapse to a truncated form.

FIG. 7D shows the Cas9 and gRNA expression cassettes, which are similar to the cassettes used in FIGS. 3B and 3C.

FIGS. 4A, 4B, 4D, and 4E illustrate basic components of the system including Cas9, gRNA, and scratchpads, while FIGS. 7C and 7D provide more details. FIGS. 4C and 4F illustrate how mutations can be used to infer/reconstruct lineage trees. FIG. 7E also illustrates this same concept. Barcoded scratchpads are mutated over time, and the patterns of shared mutations can be used to infer relatedness among cells. This figure also illustrates how the mutations can be read out by FISH (last row of figure).

Sequence information for the sample system illustrated in FIG. 7 is specifically defined in Example 2. However, one of skill in the art would understand that many sequences can be used as a guide sequence in a genetic scratchpad so long as they meet certain criteria. Exemplary criteria include 1) the sequence can function as a gRNA as defined in standard CRISPR biology, and 2) the sequence can target one or more of the homologous regions of the scratchpad.

In some embodiments, a Cas9/gRNA targeted scratchpad that operates through scratchpad collapse is provided. As disclosed herein, the system can include any sequence composed of repeating sequence segments. In other embodiments, the system can include any sequence with at least 2 homologous regions that are more than 5 base pairs in length. Alternatively, the homologous regions can be more than 8 bp, more than 10 bp, more than 12 bp, more than 15 bp, more than 20 bp, more than 25 bp, more than 30 bp, or more than 50 bp in length.

In some embodiments, the system can include scratchpad sequences that are targeted by other systems beyond Cas9/gRNA, such as a nuclease, recombinase, integrase, and etc. Another nuclease might be able to use the Cas9/gRNA scratchpad design principles as described above. A recombinase or integrase will require a scratchpad sequence that includes recognition sequences specific to the enzyme. The embodiments here are provided by way of example and should not in any way limit the scope of the invention. As disclosed herein, the scratchpad sequence undergoes a mutation upon being targeted and the mutation is detectable by a detection method such as FISH, gel electrophoresis, and/or sequencing.

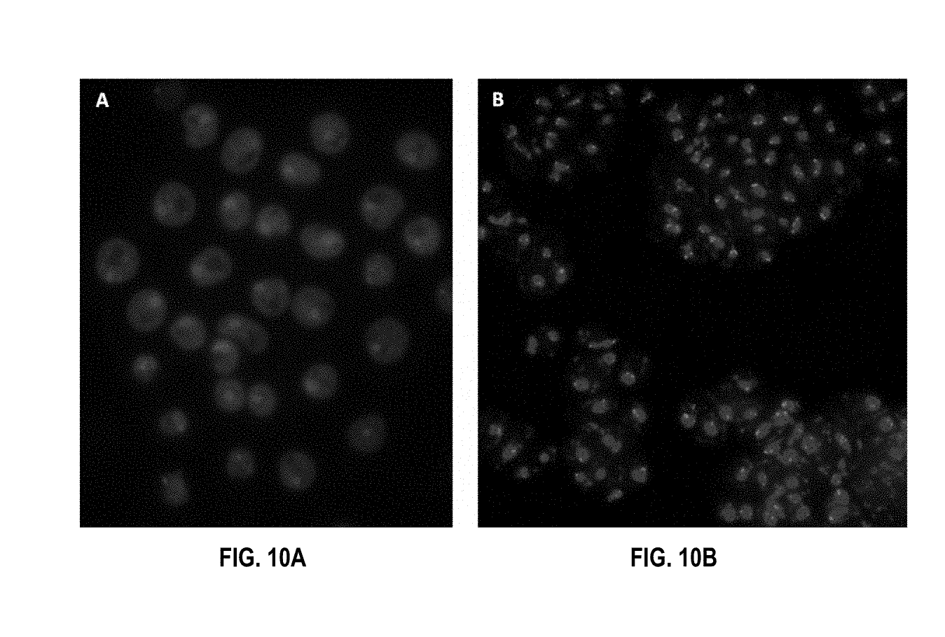

In some embodiments, the system disclosed herein is used to record lineage of non-mammalian cells such as yeast cells (e.g., FIG. 10B). In some embodiments, the system disclosed herein is used to record lineage of mammalian cells such as mouse embryonic stem cells (e.g., E14); see, for example, FIGS. 10A, 11A, 11B, 18).

In some embodiments, the system disclosed herein can also be implemented in organisms, including but not limited to, for example, mice, zebrafish, and flies. For example, engineered ES cells can be used to make transgenic or chimeric embryos or animals. For example, mESC can be used to populate a mouse embryo to make a chimeric embryo/mouse and ultimately to make mice harboring this system. Therefore, the engineering mESCs developed herein can be directly used to “make a mouse.”

Beyond lineage analysis, the system and method described herein has many additional applications. This technology disclosed herein is very useful for the study of cell development/differentiation and disease genesis or progression.

In some embodiments, the system and method can be used to study differentiation of stem cells in order to track the lineage relationships of stem cells that differentiate into different states/cell types. In some embodiments, the system and method can be used to study differentiation of stem cells in order to record which developmental signals cause cells to adopt different cell fates.

In some embodiments, the system and method can be used as lineage tracking during the development of an organism (e.g., a mouse or other organisms) to understand the lineage relationships of cells that ultimately form different organs, e.g., the brain. In some embodiments, the system and method can be used to record cellular events that happen during cell fate specification in developing mouse (or other organisms) embryos, e.g., signal 1 and then signal 2 are required for a cell to adopt fate X.

In some embodiments, a cell line that can be used in the current system includes but is not limited to C8161, CCRF-CEM, MOLT, mIMCD-3, NHDF, HeLa-S3, Huh1, Huh4, Huh7, HUVEC, HASMC, HEKn, HEKa, MiaPaCell, Panel, PC-3, TF1, CTLL-2, C1R, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bc1-1, BC-3, IC21, DLD2, Raw264.7, NRK, NRK-52E, MRCS, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-MeI 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof.

In some embodiments, a cell line that can be used in the current system includes but is not limited to HeLa cell, Chinese Hamster Ovary cell, 293-T cell, a pheochromocytoma, a neuroblastomas fibroblast, a rhabdomyosarcoma, a dorsal root ganglion cell, a NSO cell, Tobacco BY-2, CV-I (ATCC CCL 70), COS-1 (ATCC CRL 1650), COS-7 (ATCC CRL 1651), CHO-K1 (ATCC CCL 61), 3T3 (ATCC CCL 92), NIH/3T3 (ATCC CRL 1658), HeLa (ATCC CCL 2), C 1271 (ATCC CRL 1616), BS-C-I (ATCC CCL 26), MRC-5 (ATCC CCL 171), L-cells, HEK-293 (ATCC CRL1573) and PC 12 (ATCC CRL-1721), HEK293T (ATCC CRL-11268), RBL (ATCC CRL-1378), SH-SY5Y (ATCC CRL-2266), MDCK (ATCC CCL-34), SJ-RH30 (ATCC CRL-2061), HepG2 (ATCC HB-8065), ND7/23 (ECACC 92090903), CHO (ECACC 85050302), Vera (ATCC CCL 81), Caco-2 (ATCC HTB 37), K562 (ATCC CCL 243), Jurkat (ATCC TIB-152), Per.Có, Huvec (ATCC Human Primary PCS 100-010, Mouse CRL 2514, CRL 2515, CRL 2516), HuH-7D12 (ECACC 01042712), 293 (ATCC CRL 10852), A549 (ATCC CCL 185), IMR-90 (ATCC CCL 186), MCF-7 (ATC HTB-22), U-2 OS (ATCC HTB-96), and T84 (ATCC CCL 248), or any cell available at American Type Culture Collection (ATCC), or any combination thereof.

In some embodiments, any cell type derived from the above cell lines can be used. For example, mESC can be differentiated to give different types of cells (such as neurons, smooth muscles and etc.).

The methods and systems disclosed herein are also ideal for applications beyond lineage tracking, including event recording in single cells and tissues. By using multiple variants of scratchpads and writing components, different types of events can be recorded in parallel. And, this method makes it possible to resolve the timing of these events by using lineage tracking principles to map inherited mutations backward in time. Transcriptional, signaling, and other cellular events can be recorded in the genome. Ultimately, this history can be read out and the cell's or tissue's history reconstructed.

In some embodiments, the methods and systems disclosed herein can be used to record events leading to tumorigenesis or metastasis in tissue and animal models, thereby facilitating understanding of mechanisms underlying tumor formation or migration. In some embodiments, the impact of treatments identified to disrupt tumor genesis or metastasis can be assessed with this same approach.

In some embodiments, the methods and systems disclosed herein can use lineage tracking to study which cells populate a tumor and/or lead to tumor metastasis.

In some embodiments, the methods and systems disclosed herein can be used to record events that trigger the development of disease in a tissue, such as the events that lead to tumorigenesis or metastasis in certain cells. For example, the in situ readout capability of the current system allows mapping of cell relatedness and cell state spatially within a tumor, allowing one to connect growth, invasion, and metastasis to physical features of the tumor. the current system can be implemented in established models of metastasis, such as the 4T1 mammary cell line. The current system will produce in vivo, high resolution lineage map that not only provide a unique view of the dynamics of breast tumor formation, but address long standing questions regarding the origin of metastasis from the primary breast tumor and the timing of key events in the progression to metastasis.

Importantly and uniquely, the system can be used in situ to provide information on cells in their native context. This allows one to get lineage and molecular event information on tissues without disrupting them. The anatomy of tissues and organs can, therefore, be probed without loss of critical spatial information. For example, to understand tumor metastasis, it is important to consider the anatomy of the original tumor and its metastases.

As disclosed herein, the current system and method can be applied to analyze diseases or disorders including but not limited to: Neoplasia, Age-related Macular Degeneration, Schizophrenia, Trinucleotide Repeat Disorders, Fragile X Syndrome, Secretase Related disorders, Others Prior-related disorders, ALS, Drug addiction, Autism, Alzheimer's Disease, Inflammation, Blood and coagulation diseases, Cell dysregulation and oncology diseases and etc.

As disclosed herein, the current system and method can be applied to analyze cell development/differentiation by monitoring cellular functions and/or processes that include but are not limited to: PI3K/AKT Signaling, ERK/MAPK Signaling, Glucocorticoid Receptor Signaling, Axonal Guidance Signaling, Ephrin Receptor Signaling, Actin Cytoskeleton Signaling, Huntington's Disease Signaling, Apoptosis Signaling, B Cell Receptor Signaling, Leukocyte Extravasation Signaling, Integrin Signaling, Acute Phase Response Signaling, PTEN Signaling, p53 Signaling, Aryl Hydrocarbon Receptor Signaling, Xenobiotic Metabolism Signaling, SAPK/JNK Signaling, PPAr/RXR Signaling, NF-KB Signaling, Neuregulin Signaling, Wnt & Beta catenin Signaling, Insulin Receptor Signaling, IL-6 Signaling, hepatic Cholestasis, IGF-1 Signaling, NRF2-mediated Oxidative Stress Response, Hepatic, Fibrosis/Hepatic Stellate Cell Activation, PPAR Signaling, Fc Epsilon RI Signaling, G-Protein Coupled Receptor Signaling, Inositol Phosphate Metabolism, PDGF Signaling, VEGF Signaling, Natural Killer Cell Signaling, T Cell Receptor Signaling, FGF Signaling, GM-CSF Signaling, Chemokine Signaling, IL-2 Signaling and many more.

Additional examples of cell lines, cellular functions, diseases, disorders, and target sequences (e.g., including nucleic acid and protein sequences) can be found in, for example, U.S. Pat. No. 8,697,359 (e.g., Table A, Table B, Table C); U.S. Pat. No. 8,945,839; US Pat. Pub. No. 2010/0047261A1; US Pat. Pub. No. 2010/0305188A1; US Pat. Pub. No. 2014/0068797; U.S. Pat. No. 9,260,752; each of which is hereby incorporated by reference in its entirety.

In some embodiments, the methods and systems disclosed herein are used to identify one or more triggering events for tumor genesis or metastasis. In particular, in some embodiments, it is possible to identify signaling events that give rise to oncogenesis. For example, it is established that gRNA expression can be driven by promoters recognized by RNA polymerase II, therefore, signaling events that give rise to gene expression can also be used to express specific gRNAs. By coupling signal dependent mutagenesis, to a constitutive rate of mutagenesis, as described above, one will be able to identify the series of pathway events that were activated within the cells of a tumor and at what point in the lineage history of the tumor those signaling events occurred.

In some embodiments, the methods and systems disclosed herein are used to identify early activation events in neural development. For example, by coupling gRNA expression to neuronal activity via an early response promoter, such as that driving cFos expression, one will be able to identify the activation history of a given progenitor by coupling the conditional mutagenesis to the constitutive mutagenesis, as described above.

In some embodiments, the methods and systems disclosed herein are used to record changes in membrane potential and activation within post-mitotic neurons and other excitable cell types. As disclosed above, one can achieve conditional gRNA expression with the use of an early response promoter. Optimal CRISPR function may be achieved by balancing gRNA efficiency with gRNA turnover, ensuring that changes in membrane potential of a predetermined strength or duration would be accompanied by mutagenesis. Furthermore, by employing multiple, differentially tuned, gRNAs with unique target recognition, one can record events arising from action potentials of various strengths and durations. Using the same approach, one can condition optimized gRNA expression to genes associated with neurodegeneration, such as Tau or beta amyloid. In this way, events would only be recorded in those neurons overexpressing these genes. Additionally, the magnitude of mutagenesis incorporated into the scratchpad in a given neuron would identify it as the possible origin of the pathogenesis.

In some embodiments, once key events and key players are identified, it is possible to design or screen for target-specific therapeutics.

### REFERENCES

1. Sulston, J. E., Schierenberg, E., White, J. G. & Thomson, J. N. The embryonic cell lineage of the nematode Caenorhabditis elegans. Dev Biol 100, 64-119, (1983).
2. Blanpain, C. & Simons, B. D. Unravelling stem cell dynamics by lineage tracing. Nat Rev Mol Cell Biol 14, 489-502
3. Solek, C. M. & Ekker, M. Cell lineage tracing techniques for the study of brain development and regeneration. Int J Dev Neurosci 30, 560-569.
4. Xu, T. & Rubin, G. M. Analysis of genetic mosaics in developing and adult Drosophila tissues. Development 117, 1223-1237 (1993).
5. Lee, T. & Luo, L. Mosaic analysis with a repressible cell marker for studies of gene function in neuronal morphogenesis. Neuron 22, 451-461, (1999).
6. Tasic, B. et al. Extensions of MADM (mosaic analysis with double markers) in mice. PLoS One 7, e33332.
7. Livet, J. et al. Transgenic strategies for combinatorial expression of fluorescent proteins in the nervous system. Nature 450, 56-62.
8. Levesque, M. J., Ginart, P., Wei, Y. & Raj, A. Visualizing SNVs to quantify allele-specific expression in single cells. Nat Methods 10, 865-867.
9. Chung, K. et al. Structural and molecular interrogation of intact biological systems. Nature 497, 332-337.

Having described the invention in detail, it will be apparent that modifications, variations, and equivalent embodiments are possible without departing from the scope of the invention defined in the appended claims. Furthermore, it should be appreciated that all examples in the present disclosure are provided as non-limiting examples.

### EXAMPLES

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

### Example 1

### Materials and Methods

Recording system component construction. The scratchpad transposon was constructed from a ten-repeat array (20× PP7 stem loops) derived from plasmid pCR4-24×PP7SL and ligated directionally using BamH1 and BglII sites into a modified form of the PiggyBac (PB) vector PB510B (SBI) lacking the 3′ insulator and including a multiple cloning site (MCS). The CMV promoter was then removed using NheI and SpeI and replaced by a PGK promoter with Gibson assembly. A gBlock (IDT) containing the AvrII and XhoI restriction sites, priming sequences, and the BGH polyA was then introduced 3′ of the PP7 array by Gibson assembly using the EagI site in the backbone. Unique barcodes were then inserted into the transposon in the region 3′ of the scratchpad array either by Gibson assembly or directed ligation using AvrII and XhoI. A total of 28 unique barcode sequences (GenScript Biotech) derived from Saccharomyces cerevisiae were used to generate the barcoded scratchpads. Scratchpad transposons were found to produce transcripts with half-lives of approximately 2 h.

The Cas9 construct was made using hSpCas9 from pX330. First, the FKBP degron (DD) was PCR-amplified from pBMN FKBP(DD)-YFP14 and introduced with Gibson assembly into pX330 restricted with AgeI, 5′ of the open reading frame of hSpCas9, to create pX330-DD-hSpCas9. DD-hSpCas9 was amplified from this plasmid by PCR and introduced into another plasmid, 3′ of a PGK promoter using Gibson assembly. After sequence verification, the PGK-DD-hSpCas9 construct was excised using restriction enzymes (AvrII and SacII), blunted with T4 polymerase, and ligated into a modified form of the PiggyBac vector PB510B (SBI) lacking the CMV promoter and including a MCS. A non-transposon version of Cas9 was also created using hSpCas9 amplified from pX330 and introduced with Gibson assembly at the 3′ end of a CMV promoter containing two Tet operator sites into a standard plasmid backbone.

The Wnt-pathway-responsive gRNA expression transposon was created using a LEF-1 response element. The enhancer and promoter combination exhibited low basal activity, large dynamic range, and responsiveness to the GSK3 inhibitor CHIR99021 and the Wnt3a ligand. This Wnt sensor was cloned upstream of a nuclear localization signal (NLS)-tagged mTurquoise2, which served as a reporter of guide expression, that contained an embedded gRNA. The gRNA was flanked by self-cleaving ribozymes to excise it from the mRNA, and was purchased as a gblock (IDT) and inserted using Gibson assembly between the end of the mTurquoise2 coding sequence and a SV40 polyA. This construct was contained in a modified form of the PiggyBac vector PB510B.

The Cre-activated gRNA expression transposon was created using the U6 TATA-lox promoter design. The promoter, shRNA against mTurquoise2, and gRNA regions were purchased as a gblocks or oligos (IDT) and inserted into a modified form of the PiggyBac vector PB510B containing PGK-H2B-mTurquoise2.

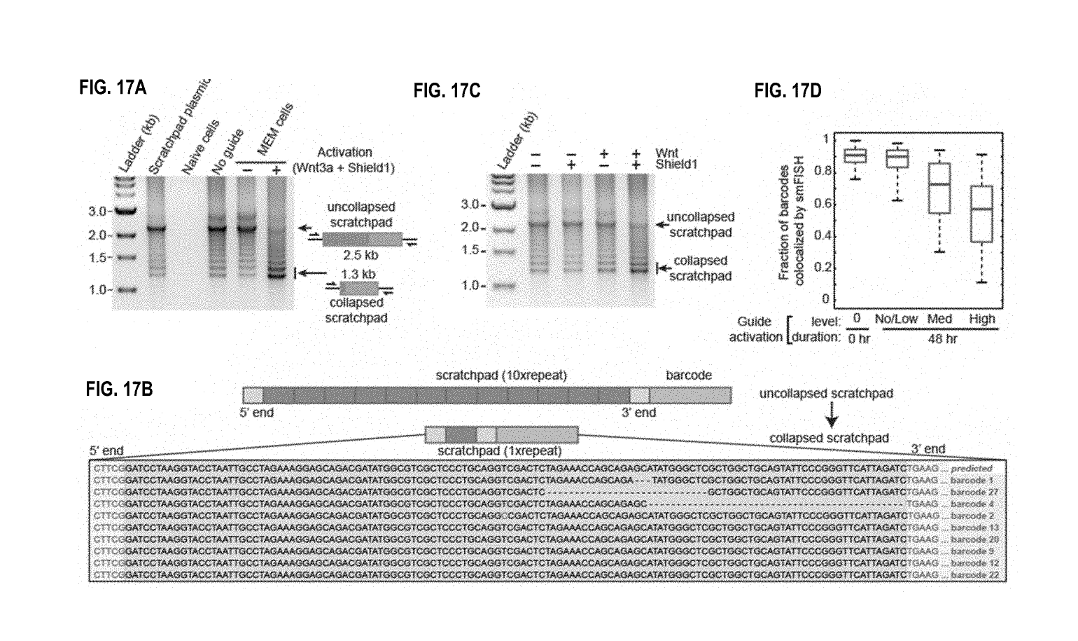

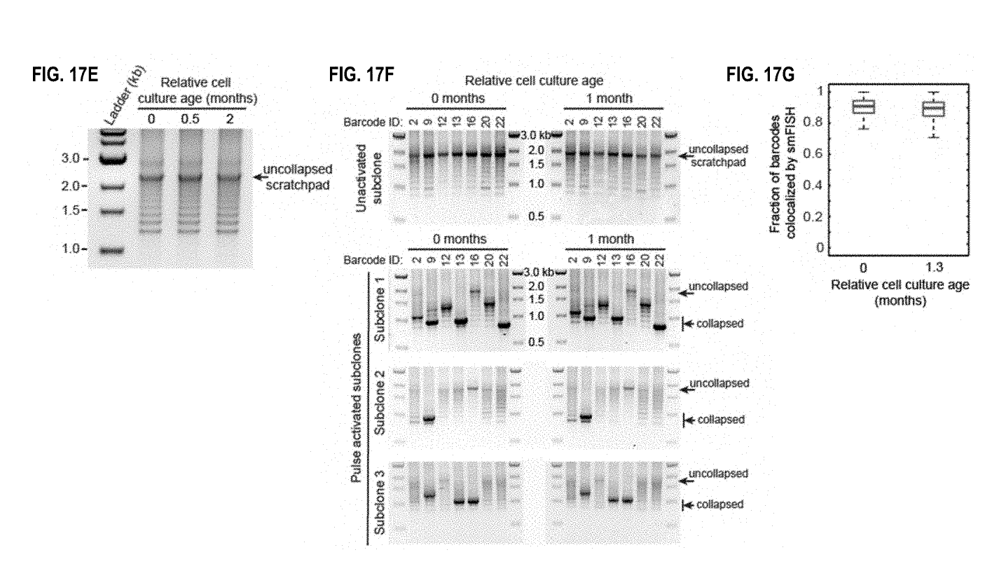

Cell line engineering and culture conditions. To create MEM-01, the E14 mouse embryonic stem cell line (ATCC cat no. CRL-1821) was co-transfected with expression plasmids for-hSpCas9 and the Tet repressor and then selected on neomycin. A single Cas9-positive clone was then used for co-transfection of 28 PB transposon barcoded scratchpads and a PB transposon PGK-palmitoylated-mTurquoise2/HygroR to facilitate segmentation of cell membranes and selection on hygromycin. Subsequent scratchpad-containing clones were inspected for overall scratchpad expression by smFISH. Scratchpad clones were also assessed for Cas9 expression, which was found to be very low and heterogeneous in most clones, with no expression in many cells (for example, 6±21 transcripts per cell). A scratchpad clone with good scratchpad expression was then simultaneously transfected with the DD-hSpCas9 PB transposon (to improve Cas9 expression (26±17 transcripts per cell)) and the Wnt-activated gRNA expression PB transposon. Cells were selected on blasticidin. Single clones were assessed for activation potential on the basis of mTurquoise2 expression in response to CHIR99021 (Stemgent) or Wnt3a (1324-WN-002 R&D systems), and enhanced Cas9 expression was measured by smFISH. Among these clones was MEM-01, which demonstrated good gRNA activation in response to Wnt3a and increased Cas9 activity in the presence of the stabilizing agent, Shield 1 (Clontech) (FIG. 17C). MEM-01 resembled the parental E14 line in terms of cell morphology, cycle times, and expression of pluripotency markers including Esrrb, Nanog, and SSEA-1. Stably selected cell lines containing a Cre-activated gRNA were similarly engineered.

The transfections described above were carried out using Fugene HD (Promega) at a mass (μg) DNA/volume (μl) Fugene ratio of 1:3 and following the manufacturer's instructions. For transfection of the PB components a total DNA mass of 1 μg was used at a ratio of 6:1, PB transposons to PB transposase PB200PA-1 (SBI). For selection with antibiotics, transfected cells were lifted with Accutase (ThermoFisher) after transfection media was removed and plated on 100-mm plates (Nunc). 24 h later growth media was replaced with selection media. Single colonies were lifted from selection plates as they matured.

During standard cell culturing, ES cells were maintained at 37° C. and 5% CO2 in GMEM (Sigma), 15% ES cell qualified fetal bovine serum (FBS) (Gibco/ThermoFisher), PSG (2 mM 1-glutamine, 100 units per ml penicillin, 100 μg per ml streptomycin) (ThermoFisher), 1 mM sodium pyruvate (ThermoFisher), 1,000 units per ml Leukaemia Inhibitory Factor (LIF, Millipore), 1× Minimum Essential Medium Non-Essential Amino Acids (MEM NEAA, ThermoFisher) and 50-100 μM β-mercaptoethanol (Gibco/ThermoFisher). Cells were maintained on polystyrene (Falcon) coated with 0.1% gelatin (Sigma).

Quantitative PCR. For detection of genomic barcode copy number, genomic DNA was prepared from cells using the DNeasy Blood and Tissue kit (Qiagen). DNA was quantified on a NanoDrop 8000 spectrophotometer (ThermoScientific). Reactions were assembled as above with around 1,000-5,000 haploid genome copies, based on 3 picograms per haploid genome approximation. For gene expression analysis, total RNA was prepared using the RNeasy Mini kit (Qiagen). One microgram of total RNA was used with the iScript cDNA synthesis kit (BioRad) following the manufacturer's instructions. For qPCR a 1:20 dilution of the cDNA was used in each reaction. All reactions were performed with IQ SYBR Green Supermix (BioRad). Reaction cycling was carried out on a BioRad CFX96 thermocycler. Both genomic DNA and cDNA samples were compared against Sdha copy number or expression level, respectively. Analyses included at least three biological replicates with each reaction run in triplicate, unless otherwise noted. Primer sets for all barcodes and normalizers were obtained from IDT, and the efficiencies of all primer pairs were tested.

Time-lapse videos and cell culture for imaging. Tissue culture grade glass bottom 24-well plates (MatTek) were treated with laminin-511 (20 μg per ml) (Biolamina) for 4 h at 37° C. and plated with cells at approximately 2,500 cells per cm2. Cells were exposed to Wnt3a (50-100 ng per ml) and Shield1 (50-100 nM) at the time of plating. After approximately 16 h, cells were selected for time-lapse imaging based on system activation, assessed by visible mTurquoise2 signal, and then imaged in an incubated microscope environment every 14 min over 20-40 h before being immediately fixed. Samples were fixed with 4% formaldehyde in PBS for 5 min. Samples cultured for smFISH imaging, but without time-lapse video tracking, were prepared similarly (typically with a higher plated cell density) and activated for different lengths of time, as stated.

Single molecule fluorescence in situ hybridization (smFISH). Hybridization and imaging were carried out with the following exceptions: scratchpad transcripts were targeted with 40 DNA oligo 20mer probes and barcode regions were targeted with 18 20mer probes. Probes were coupled to one of three dyes (Alexa 555, 594 or 647 (ThermoFisher)) and used at approximately 130 nM concentration per probe set. Post-hybridization, cells were washed in 20% formamide in 2×SSC containing DAPI at 30° C. for 30 min, rinsed in 2×SSC at room temperature, and imaged in 2×SSC. For seqFISH, after imaging each round of hybridization, 2×SSC was replaced with wash buffer for about 5 min at room temperature and then replaced with the next probe set in hybridization buffer for overnight incubation. Most barcode signals from the previous hybridization were no longer visible during imaging of the following hybridization (owing to photobleaching and probe loss facilitated by the small number of barcode probes (18) used per barcode); any remaining visible transcripts were computationally subtracted during analysis. Incubation, washing, and imaging proceeded as above for up to nine rounds of hybridization.

For analysis of smFISH images, semi-automated cell segmentation and dot detection were performed using custom Matlab software. Raw images were processed by a Laplacian of the Gaussian filter and then thresholded to select dots. Co-localization between dots in the scratchpad image and barcode image was detected if both dots were above the threshold and within a few pixels of each other. To generate the histogram of intensities for the collapsed and uncollapsed scratchpads in FIG. 15B, we integrated the fluorescence intensities in the regions of the scratchpad smFISH image that corresponded to individual barcode dots or the detected scratchpad dots, respectively. For the collapse rate experiment in FIG. 15C, we measured the aggregate smFISH scratchpad co-localization levels for four highly expressed barcodes in cells that had been induced for different lengths of time. For activating conditions shown in FIGS. 15B and 15C, only data from cells that were actually activated (as assessed by mTurquoise2 expression) were included.

Lineage reconstruction of experimental data. Cell-to-cell barcode distance scores were determined for each pair of cells based on the similarity of the two cells' co-localization fractions for each barcode and weighted by the barcode's transcript number (as a measure of confidence in the observation).

Lineage trees were reconstructed from the cell-to-cell barcode distance matrices using a modified version of a standard agglomerative hierarchical clustering algorithm34. Reconstructions were constrained to binary trees such that cells were paired into sisters before first cousin pairs were assigned. Pairing proceeded by successively grouping pairs of cells or cell clusters with the minimum barcode distance. At each step, if the two most optimal (that is, minimum distance) pairings were close in distance, the algorithm optimized for the lowest combined distance of the current and next minimum distances. The distance between two clusters was computed using the standard UPGMA algorithm19 by averaging the cell-to-cell barcode distance between all possible pairs of cells across the two clusters.

Bootstrap to identify robust reconstructions. For each colony, the barcoded scratchpad data were resampled by bootstrap and corresponding lineage trees were reconstructed (n=1,000 resampled reconstructions per colony). On the basis of the frequency at which the original cousin clades occurred in the resampled reconstructed trees, a robustness score was assigned to each colony. Colonies whose clade reconstructions were less sensitive to resampling showed significantly improved overall reconstruction accuracy. Subsets of colonies with more reliable reconstructions could thus be selected without prior knowledge of their accuracy by selecting colonies with higher robustness scores, for example, scores in the top 20-40% of the data.

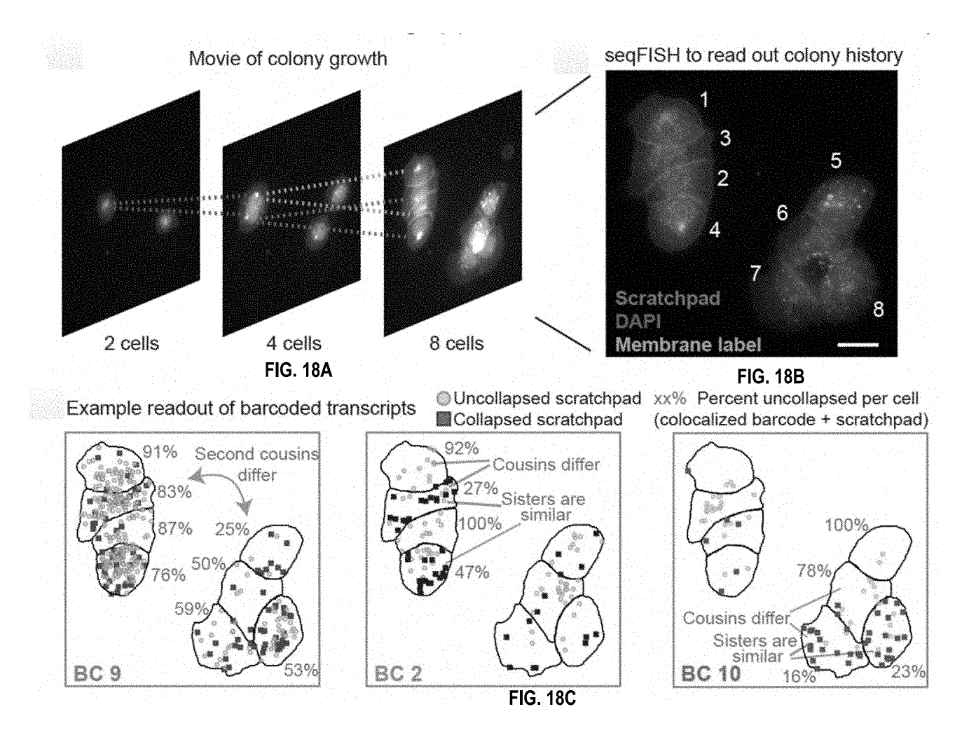

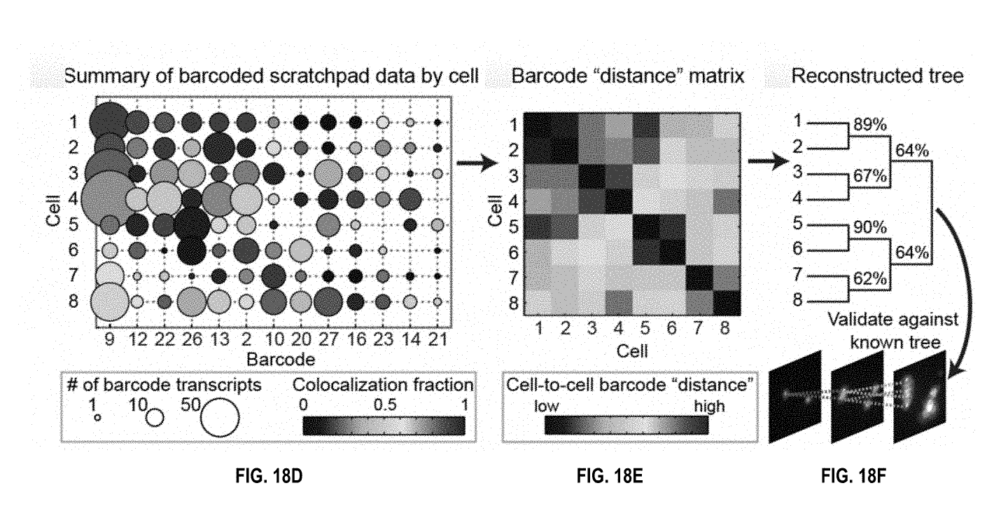

Alternative metrics for identifying colonies with robust lineage information were also tested. These metrics similarly enriched for subsets of data with improved reconstruction accuracy, further supporting the observation that some colonies showed clear lineage information while others did not acquire well-defined collapse patterns, probably owing to limited, excessive, or ambiguous collapse events. Lineage reconstruction simulations. To simulate the recording for three-generation binary trees, experiments were started with one cell with a fixed number of idealized scratchpads. At each division, the daughter cells inherited the same scratchpad profile as their parent and independently collapsed each uncollapsed site with a fixed probability, defined as the collapse rate. After three generations, the scratchpad profiles of the eight resulting cells were used to reconstruct their lineage tree using either a modified neighbor joining algorithm, or the Camin-Sokal maximum parsimony algorithm35 that exhaustively scored all 315 possible tree reconstructions. Both forward simulations and the reconstruction algorithms were implemented in Matlab. For the heat map and the cumulative distribution functions, the fraction of correct relationships was computed as the fraction of all distinct pairwise relationships in the actual tree that were correctly identified in the reconstructed tree. If multiple reconstructions were equally valid (same parsimony score), the fraction of correct relationships was averaged over all of them. Reconstruction accuracy was tested over a wide range of collapse rates or for the approximate collapse rate observed in our experiments, 0.1 per site per generation. The empirical collapse rate, 0.1, was estimated from the observed co-localization fraction of the barcodes, ˜0.67, in 108 MEM-01 colonies induced for approximately 48 h (same colonies as in FIG. 18). Additionally, trees of a higher number of generations were reconstructed from the final collapse pattern using a modified neighbour joining algorithm in which allowed reconstructions were restricted to full binary trees (data not shown). Fraction of correct relationships was again computed as the fraction of all distinct pairwise relationships in the actual tree that were correctly identified in the reconstructed tree averaged over at least 1,000 trees.

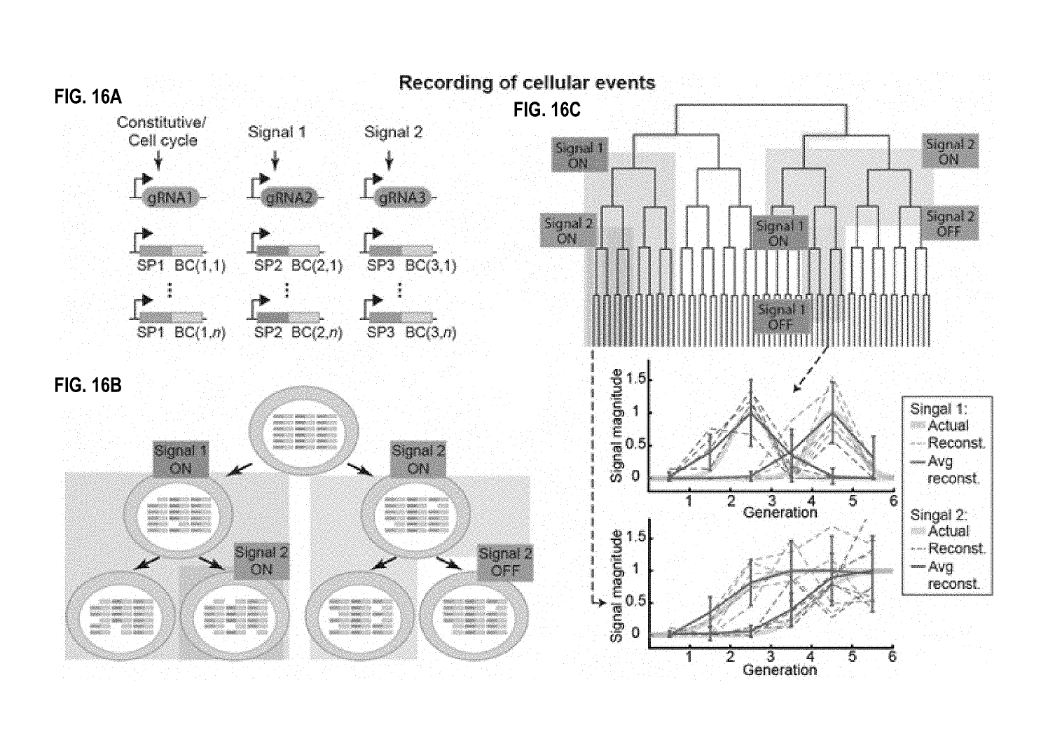

Event recording simulations. Simulation of signal recording. Demonstrations of event recording were simulated isomg the same forward tree-generation algorithm as in the exemplary lineage reconstruction simulations, for trees of six generations, assuming 50 idealized scratchpads and a collapse rate of 0.1 per scratchpad per generation. The simulated cells also contained two additional sets of recording scratchpads of 50 sites each (FIG. 16A). It is assumed these scratchpads collapsed through independent events occurring at rates proportional to the magnitude of their respective input signals. The minimum and maximum collapse rates at low and high signal were set to 0 and 0.2 per scratchpad per generation, respectively. The magnitude of the input signals varied over time and from branch to branch as shown in FIGS. 16B and 16C, resulting in different collapse rates for each of the two recording scratchpad sets over time and along different lineages. FIGS. 16A-16C correspond to the schematic representation of FIGS. 6A-6C. For example, FIG. 16A is highly similar to FIGS. 6A and 6B except that the gRNA and barcode/target sequences are specified in FIG. 16A. Similarly, FIG. 6C is similar to FIGS. 16B and 16C, except that the latter provides more details.

Reconstruction of simulated signal dynamics. The lineage tree was first reconstructed using only the lineage-tracking scratchpad sites. This reconstruction used a neighbor-joining algorithm. The reconstructed history of the collapse events of the recording scratchpads was then mapped onto the reconstructed lineage tree. For this procedure, a Camin-Sokal maximum parsimony algorithm was employed. In brief, the algorithm proceeds from the leaves of the tree to the root. At each generation, it infers the collapse state of the parental node, based on the known collapse states of the two daughters, while minimizing the number of new collapse events occurring between the parent and the daughters. For binary scratchpads this corresponds to computing the intersection between the collapse patterns of the two daughters. This procedure is then repeated for the parent and its sister until reaching the root. At the end of this procedure, one obtains a maximum parsimony assignment of scratchpad states to each node in the tree. On the basis of these assignments, the number of scratchpad collapse events in recording scratchpads that occurred along each branch was calculated. Finally, this reconstructed collapse level provides an estimate of the underlying signal intensity along each lineage (for example, actual and reconstructed signals shown for two lineages of interest in FIG. 16C).

### Example 2

### Exemplary Scratchpad

Using a system illustrated in FIG. 7, the state of this scratchpad can be stochastically altered in live cells and read out in situ in single cells by smFISH. In this example, the scratchpad element consisted of 10 repeat units. gRNA targeting of Cas9 to the scratchpad generated double-strand breaks that result in its deletion, cut or ‘collapse’. (see e.g., FIGS. 7C and 7D, 8A, 8B, 17A, 17F). Adjacent to each scratchpad, a co-transcribed barcode was incorporated. The barcode and scratchpad components was each be identified using specific sets of smFISH probes, and thus served as an addressable ‘bit’.

Using a pool of such barcoded scratchpads enables lineage recording and readout through a two-step process. During cell proliferation, Cas9 generates gradual and stochastic accumulation of collapsed scratchpads in each cell lineage. Subsequently, cells can be fixed and analyzed by seqFISH to identify barcodes and assess their states based on the presence or absence of a co-localized scratchpad signal (FIG. 7E).

To implement the sample recording system, a stable mouse embryonic stem (ES) cell line (designated MEM-01) was engineered, which incorporated barcoded scratchpads, Cas9, and a scratchpad-targeting gRNA (FIG. 7D). First, PiggyBac transposition was used to integrate a set of 28 barcoded scratchpad elements into the genome. A clone was identified in which 13 different barcodes were highly expressed. Within this line, a Cas9 variant containing an inducible degron was stably integrated to allow external modulation of Cas9 activity. Finally, a scratchpad-targeting gRNA expressed from a Wnt-regulated promoter was engineered, to enable both external control as well as recording of Wnt pathway activity.

In the example illustrated in FIGS. 7C and 7D, a PGK promoter sequence was used. The Cas9 expression cassette, gRNA expression cassette and scratchpads were introduced as transposons into the genome of the cell using the PiggyBac transposon system and standard transfection techniques. The scratchpad was a 10 repeat array of a bacteria phage PP7 sequence. The protospacer element used as the gRNA target sequence in this example was:

GTAGAAACCAGCAGAGCATA

Sequence information for the PP7 repeats can be found below.

PP7 repeated unit:
TAAGGTACCTAATTGCCTAGAAAGGAGCAGACGATATGGCGTCGCTCCCT
GCAGGTCGACTCTAGAAACCAGCAGAGCATATGGGCTCGCTGGCTGCAGT
ATTCCCGGGTTCATT
Scratchpad array of 10 PP7 repeats (1210 bp):
GATCCTAAGGTACCTAATTGCCTAGAAAGGAGCAGACGATATGGCGTCGC
TCCCTGCAGGTCGACTCTAGAAACCAGCAGAGCATATGGGCTCGCTGGCT
GCAGTATTCCCGGGTTCATTAGATCCTAAGGTACCTAATTGCCTAGAAAG
GAGCAGACGATATGGCGTCGCTCCCTGCAGGTCGACTCTAGAAACCAGCA
GAGCATATGGGCTCGCTGGCTGCAGTATTCCCGGGTTCATTAGATCCTAA
GGTACCTAATTGCCTAGAAAGGAGCAGACGATATGGCGTCGCTCCCTGCA
GGTCGACTCTAGAAACCAGCAGAGCATATGGGCTCGCTGGCTGCAGTATT
CCCGGGTTCATTAGATCCTAAGGTACCTAATTGCCTAGAAAGGAGCAGAC
GATATGGCGTCGCTCCCTGCAGGTCGACTCTAGAAACCAGCAGAGCATAT
GGGCTCGCTGGCTGCAGTATTCCCGGGTTCATTAGATCCTAAGGTACCTA
ATTGCCTAGAAAGGAGCAGACGATATGGCGTCGCTCCCTGCAGGTCGACT
CTAGAAACCAGCAGAGCATATGGGCTCGCTGGCTGCAGTATTCCCGGGTT
CATTAGATCCTAAGGTACCTAATTGCCTAGAAAGGAGCAGACGATATGGC
GTCGCTCCCTGCAGGTCGACTCTAGAAACCAGCAGAGCATATGGGCTCGC
TGGCTGCAGTATTCCCGGGTTCATTAGATCCTAAGGTACCTAATTGCCTA
GAAAGGAGCAGACGATATGGCGTCGCTCCCTGCAGGTCGACTCTAGAAAC
CAGCAGAGCATATGGGCTCGCTGGCTGCAGTATTCCCGGGTTCATTAGAT
CCTAAGGTACCTAATTGCCTAGAAAGGAGCAGACGATATGGCGTCGCTCC
CTGCAGGTCGACTCTAGAAACCAGCAGAGCATATGGGCTCGCTGGCTGCA
GTATTCCCGGGTTCATTAGATCCTAAGGTACCTAATTGCCTAGAAAGGAG
CAGACGATATGGCGTCGCTCCCTGCAGGTCGACTCTAGAAACCAGCAGAG
CATATGGGCTCGCTGGCTGCAGTATTCCCGGGTTCATTAGATCCTAAGGT
ACCTAATTGCCTAGAAAGGAGCAGACGATATGGCGTCGCTCCCTGCAGGT
CGACTCTAGAAACCAGCAGAGCATATGGGCTCGCTGGCTGCAGTATTCCC
GGGTTCATTA

Another example of a sequence of repeating elements is the MS2 repeat sequence.

MS2 repeat sequence:
GATCCTACGGTACTTATTGCCAAGAAAGCACGAGCATCAGCCGTGCCTCC
AGGTCGAATCTTCAAACGACGACGATCACGCGTCGCTCCAGTATTCCAGG
GTTCATC
MS2 full sequence:
GATCCTACGGTACTTATTGCCAAGAAAGCACGAGCATCAGCCGTGCCTCC
AGGTCGAATCTTCAAACGACGACGATCACGCGTCGCTCCAGTATTCCAGG
GTTCATCAGATCCTACGGTACTTATTGCCAAGAAAGCACGAGCATCAGCC
GTGCCTCCAGGTCGAATCTTCAAACGACGACGATCACGCGTCGCTCCAGT
ATTCCAGGGTTCATCAGATCCTACGGTACTTATTGCCAAGAAAGCACGAG
CATCAGCCGTGCCTCCAGGTCGAATCTTCAAACGACGACGATCACGCGTC
GCTCCAGTATTCCAGGGTTCATCAGATCCTACGGTACTTATTGCCAAGAA
AGCACGAGCATCAGCCGTGCCTCCAGGTCGAATCTTCAAACGACGACGAT
CACGCGTCGCTCCAGTATTCCAGGGTTCATCAGATCCTACGGTACTTATT
GCCAAGAAAGCACGAGCATCAGCCGTGCCTCCAGGTCGAATCTTCAAACG
ACGACGATCACGCGTCGCTCCAGTATTCCAGGGTTCATCAGATCCTACGG
TACTTATTGCCAAGAAAGCACGAGCATCAGCCGTGCCTCCAGGTCGAATC
TTCAAACGACGACGATCACGCGTCGCTCCAGTATTCCAGGGTTCATCAGA
TCCTACGGTACTTATTGCCAAGAAAGCACGAGCATCAGCCGTGCCTCCAG
GTCGAATCTTCAAACGACGACGATCACGCGTCGCTCCAGTATTCCAGGGT
TCATCAGATCCTACGGTACTTATTGCCAAGAAAGCACGAGCATCAGCCGT
GCCTCCAGGTCGAATCTTCAAACGACGACGATCACGCGTCGCTCCAGTAT
TCCAGGGTTCATCAGATCCTACGGTACTTATTGCCAAGAAAGCACGAGCA
TCAGCCGTGCCTCCAGGTCGAATCTTCAAACGACGACGATCACGCGTCGC
TCCAGTATTCCAGGGTTCATCAGATCCTACGGTACTTATTGCCAAGAAAG
CACGAGCATCAGCCGTGCCTCCAGGTCGAATCTTCAAACGACGACGATCA
CGCGTCGCTCCAGTATTCCAGGGTTCATCAGATCCTACGGTACTTATTGC
CAAGAAAGCACGAGCATCAGCCGTGCCTCCAGGTCGAATCTTCAAACGAC
GACGATCACGCGTCGCTCCAGTATTCCAGGGTTCATCAGATCCTACGGTA
CTTATTGCCAAGAAAGCACGAGCATCAGCCGTGCCTCCAGGTCGAATCTT
CAAACGACGACGATCACGCGTCGCTCCAGTATTCCAGGGTTCATCA

### Example 3

### CRISPR System Deletes Portions of Genetic Scratchpads

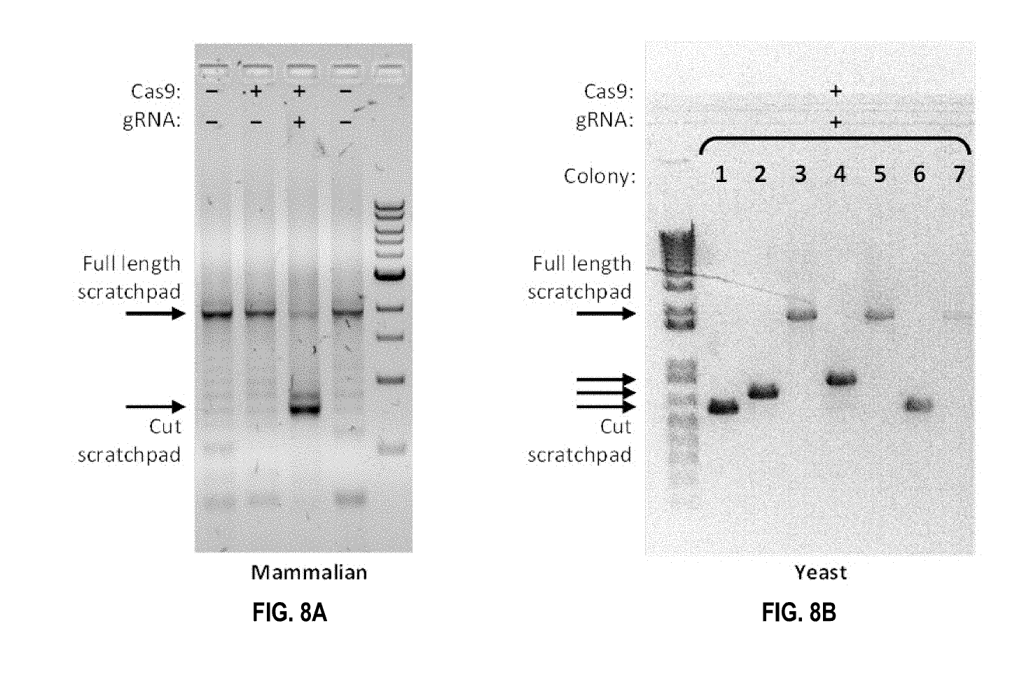

FIGS. 8A and 8B demonstrate that the CRISPR system can write on a genetic scratchpad and results in deletions of portions of sequences of the scratchpad.

FIG. 8A shows the result of bulk PCR of scratchpad in mammalian cells. Scratchpad remains intact in the absence of both gRNA and Cas9, but can be deleted when Cas9 and gRNA are both expressed. A band representing cut scratchpads is clearly visible when both gRNA and Cas9 are present, but absent when either component is missing.

FIG. 8B shows the results of individual yeast clones analysis. Here, efficient removal by the CRISPR system of most repeats of a repetitive scratchpad core is clearly observed, as indicated by multiple bands corresponding to loss of repetitive sequences from a scratchpad core. This writing approach is applicable in many organisms, including mammalian and yeast cells.

### Example 4

### Tuning of CRISPR System

This example illustrates that the cutting efficiency of Cas9 protein in the CRISPR system can be adjusted. As part of this system, Cas9 activity can be tuned through a variety of promoters, mutations, and accessory peptide fusions.

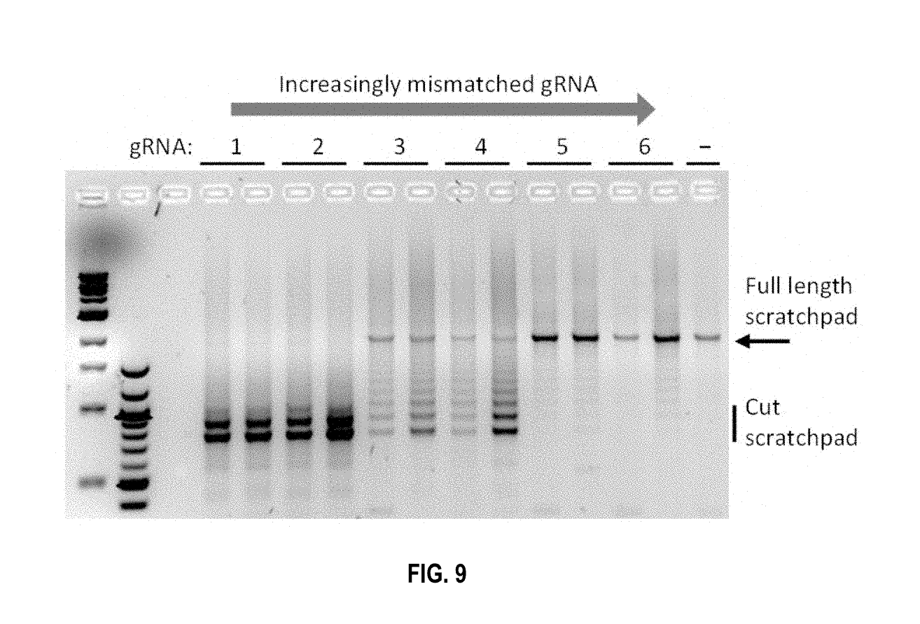

Guide RNAs can also be tuned through the use of mismatched gRNA sequences (FIG. 9), the presence of decoy gRNA, gRNA copy number control, gRNA expression from inducible promoters, and gRNA expression from atypical geometries, such as from introns. Writing can also be achieved via other systems that can alter the DNA scratchpad, including recombinase and integrase enzymes.

As shown in FIG. 9, mismatched gRNAs are one way to tune the rate of scratchpad cutting with the CRISPR system. Mismatched gRNA are not fully complementary to their target site and alter the efficiency of scratchpad cutting. gRNA less complementary to their scratchpad target show reduced (or no) cutting efficiency via bulk PCR.

### Example 5

### In Situ Characterization of Scratchpad and Mutation Status

Our method is ideal for in situ readout of events from individual cells or tissues. By using RNA FISH, we are able to visualize changes in the transcribed DNA that result from our multiple recorded events.

One implementation of this involves transcription of scratchpads from their promoters and subsequent labeling of these nascent transcripts via RNA FISH. The presence or absence (if deletion occurred) of each scratchpad as well as its uniquely identifying downstream barcode region (FIGS. 10 and 11) were visualized.

FIGS. 10A and 10B show scratchpads visualized by FISH in single cells. In FIG. 9A, a colony of mouse embryonic stem cells (red nuclei) that grew from a single cell show RNA FISH images of the scratchpad transcript (blue; seen here as one large dot). In FIG. 9B, yeast cells (blue nuclei) also show scratchpad transcripts (pink) by FISH.

FIGS. 11A and 11B illustrate scratchpad deletion observed by FISH. In both 10A and 10B, in cells lacking gRNA expression, scratchpad transcripts continue to be observed by FISH (blue dots). However, in cells transfected with a strong gRNA (identified by a co-transfection marker (green)), scratchpad transcripts (blue) are no longer present.

### Example 6

### Single Cell Scratchpad Analysis

In this example, single cell scratchpad changes read out by FISH are used to accurately reconstruct of lineage trees.

FIG. 12A shows snapshots from a movie of ES cell colony formation. The bright cell in the top left image underwent three rounds of division, resulting in eight cells. These cells contained scratchpads, Cas9, and gRNA that targeted the scratchpads for deletion over time. FIG. 12B shows the images of the final colony (green cells) by FISH of scratchpad transcripts (blue), which were used to identify cells that retained or lost scratchpads. Four of the eight cells in this colony lost their scratchpads. Based on this information, these four cells most likely underwent a scratchpad deletion event in their common ancestor and are cousins belonging to a subclade of that ancestor.

FIG. 12C shows the schematic of the maximum likelihood lineage tree inferred from FISH observations in these eight cells. The accuracy of this tree can be confirmed here by comparison with the lineage directly observed for these cells in their colony formation movie (A, most frames not shown).

### Example 7

### Sequential Barcoding to Multiplex RNA Detection in Single Cells

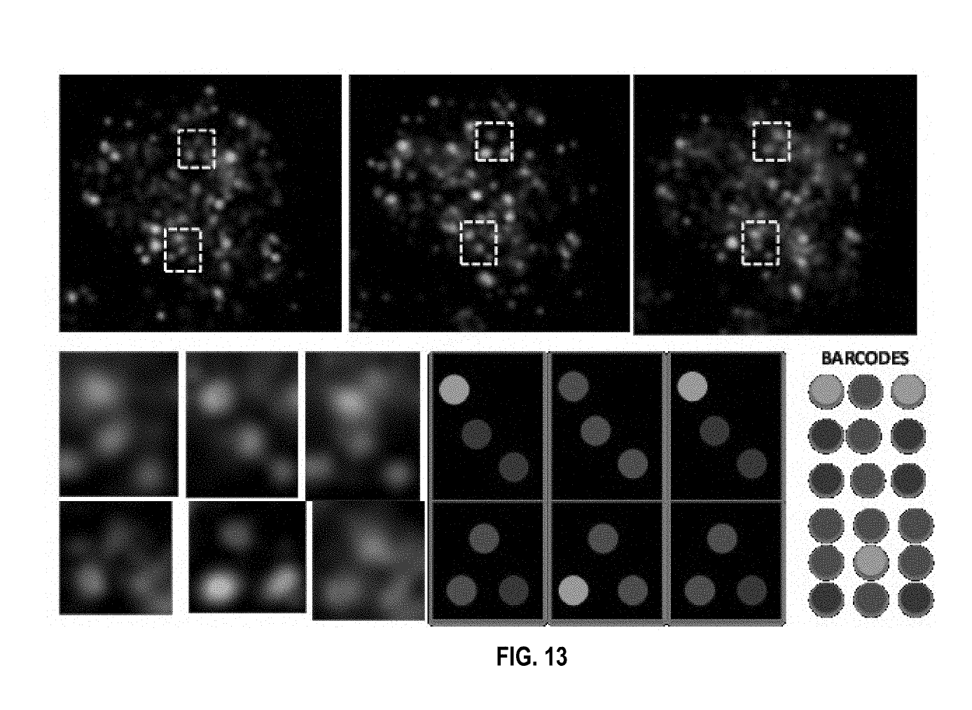

This example includes experimental data demonstrating successful sequential barcoding of transcripts in single cells, as described schematically in FIGS. 4A through 4C. Referring to FIG. 13, each dot corresponds to a distinct mRNA molecule in the cell. Three images (top left to right) show three rounds of hybridization: Hyb1, Hyb2 and Hyb3. Both Hyb1 and Hyb3 used the same labeled probes so dots colocalize, as shown in the lower panels. The lower left panel shows the zoomed in boxed region and the extracted barcodes, represented on the right, demonstrating co-localization of signals. Bottom right panels indicate interpretations of corresponding lower left panels.

### Example 8

### Simulated Recording and Multi-Generation Lineage Reconstruction

This example shows that accurate and robust algorithms can be used to reconstruct the lineage tree from a field of cells with mutagenized recording regions.

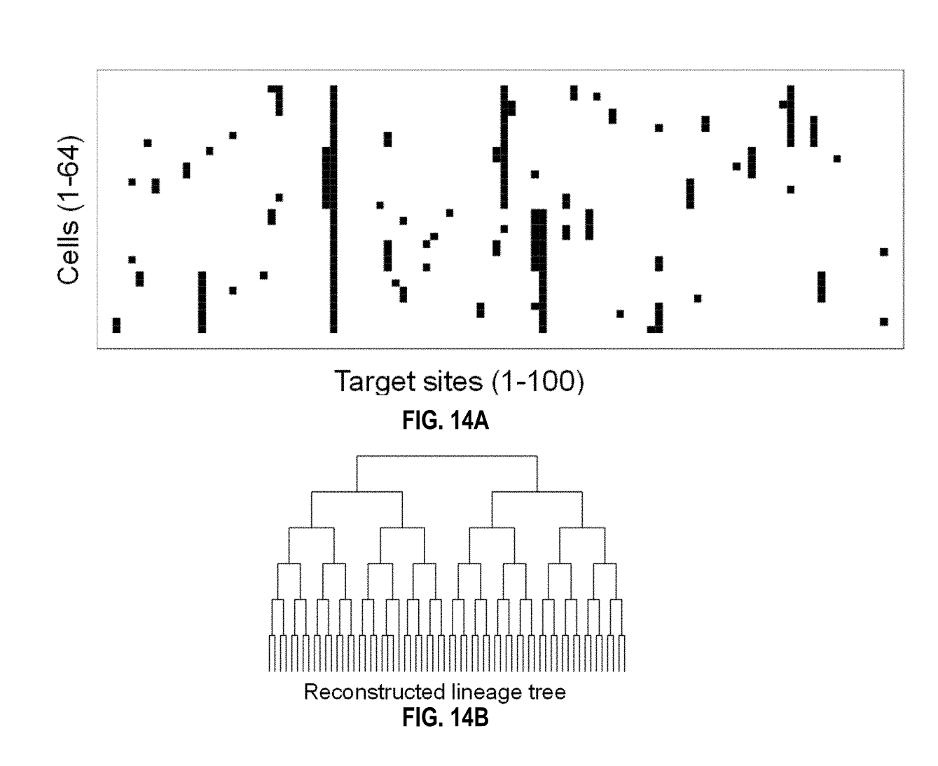

Without the spatial information on cells, computer simulation showed that 100 target sites in the recording region are sufficient to faithfully generate a 10-generation deep lineage tree (FIGS. 14A and 14B). The recording region was readout in situ preserving the spatial organization of cells, it was possible to determine through additional simulations whether this provides an additional level of robustness into the reconstruction process as well as increases the number of generations that can traced with the same number of cutting sites.

FIGS. 14A and 14B shows simulated recording region cut sites and reconstruction for a 6-generation lineage tree. In FIG. 14A, one cell was propagated for 6 generations to generate 64 descendant cells (y-axis). In each generation, a random target site from target sites No. 1-100 was cut per cell (x-axis). The recording region is shown at the end of the 6 generations. Here, a black box indicates that a target site (x axis) is mutated in a given cell, (y axis). In FIG. 14B, based on the data from FIG. 14A, a lineage tree was correctly reconstructed using Manhattan distance and complete linkage models (Mathematica).

### Example 9

### Exemplary Results

This example illustrates readout data during hybridization. FIG. 15 depicts exemplary readout during one round of hybridization. FIG. 15 corresponds to FIGS. 5, 10 and 11. For example, FIG. 5 illustrates how the recorded information can be read out using multiple rounds of smFISH. FIG. 15A shows actual smFISH readout during one round of hybridization. FIGS. 15D and 15E show a schematic (15D) and actual data (15E) on how readout works over multiple rounds of hybridization. FIGS. 10A and 11A show detection of intact and mutated scratchpads by smFISH in mammalian cells. FIGS. 15A and 15E shows scratchpad detection by smFISH in more detail.

Using this cell line, it was verified that smFISH could detect scratchpad collapse. After 48 h of Cas9 and gRNA induction, a substantial loss of scratchpad smFISH signal was observed, but not barcode signal (FIG. 15A, 15B, and FIGS. 17A through 17G). By contrast, in cells in which recording was not induced, co-localization between barcode and scratchpad signals was observed in approximately 90% of the transcripts, consistent with expected smFISH accuracies (FIGS. 15B and 15C). Although individual barcoded scratchpad transcripts appeared either collapsed or uncollapsed based on co-localization, cells typically exhibited a mixture of collapsed and uncollapsed scratch-pads with the same barcode owing to the existence of multiple genomic integrations undergoing independent collapse events. Together, these results indicate that scratchpad states can be altered and that the fraction of collapsed scratchpads for each barcode can be subsequently read out in situ.

The design of the current recording system provides a platform that can record and read out histories of dynamic cellular events beyond lineage information (FIGS. 16A and 16B). Specifically, orthogonal gRNAs expressed from signal-specific promoters can in principle record multiple intracellular signals onto distinct sets of scratchpads. Binary trees of six generations was simulated in which different cell lineages experienced distinct time courses of two input signals (FIG. 16C). In these simulations, one gRNA variant was constitutively expressed solely to enable lineage reconstruction using one set of scratchpads. In addition, each of the signals activated expression of a corresponding gRNA variant, generating collapse events in its own specific set of 50 scratchpads, at a rate proportional to the signal magnitude. Analyzing endpoint scratchpad collapse patterns for all three sets of scratchpads, allowed reconstruction of both lineage trees and event histories (FIG. 16A-16C). This reconstruction process takes advantage of the reconstructed lineage tree to map the most likely assignment of collapse events from the signal-recording gRNAs to specific positions on the lineage tree, with a maximum possible time resolution of one cell cycle (since the sequence of collapse events within a cell cycle cannot be distinguished). Thus, over timescales of multiple cell cycles, the current system should enable analysis of the sequence, duration, and magnitude of signals along individual cell lineages (FIG. 16C).

The fraction of collapsed scratchpads increased progressively over time after Cas9 and gRNA induction, as required for recording operation. An approximately 27% decrease in mean co-localization fraction was observed after 48 h of Cas9 and gRNA induction (FIGS. 15B and 15C). Additionally, the collapse rate correlated with the level of gRNA expression, suggesting that collapse rates are tunable (FIG. 17D). By contrast, in the absence of induction, scratchpad states remained stable (FIGS. 17E-17G). Further, a Cre-activated gRNA functioned similarly to the Wnt-activated gRNA, and scratchpad collapse also occurred in CHO-K1 cells and budding yeast, suggesting that the system design can be generalized to other methods of activation and to other species. Finally, it was verified that seqFISH could enable readout of 13 distinct barcoded scratchpads in single cells using 7 rounds of hybridization (see FIGS. 15D and 15E).

FIGS. 17A through 17G illustrate how scratchpads collapse in different systems, similar to FIGS. 8A and 8B.

To analyze cell lineage, the recording system was activated and cells were grown for 3 or 4 generations, while time-lapse imaging was performed to establish an independent ‘ground truth’ lineage for later validation (FIG. 18A). The cells were then fixed and analyzed their barcoded scratch-pads by seqFISH (FIG. 18B). Altogether, 108 colonies were analyzed, including 836 cells. FIG. 18 is similar to FIG. 8, which also provides examples of recorded cell growth in mammalian cells.

Inspection of scratchpad collapse patterns revealed lineage information. For example, in one colony, barcode 9 was differentially collapsed between two 4-cell clades, showing how scratchpad collapse patterns can provide insight into lineage relationships.

To analyze lineage reconstruction more systematically, scratchpad collapse frequencies were tabulated for all probed barcodes in each colony (FIG. 18D) and used to calculate a cell-to-cell ‘distance’ matrix, representing differences in collapse patterns between each pair of cells (FIG. 18E). A binary hierarchical clustering algorithm adapted from phylogenetic analysis was then applied to these distance scores in order to reconstruct a lineage tree (FIG. 18F). Finally, as validation, each reconstructed tree was compared to the actual colony lineage obtained directly from the corresponding time-lapse video (FIG. 18A).

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Various embodiments of the invention can specifically include or exclude any of these variations or elements.

In some embodiments, the numbers expressing quantities of ingredients, properties such as molecular weight, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

What is claimed is:

1. A method for characterizing lineage information or recording molecular events among cells in a cell population, comprising:
introducing, over a time period of multiple cell cycle generations, a plurality of molecular changes in at least one of one or more genetic scratchpads in one or more cells in a cell population,
wherein the cell population comprises cells that have developed for one or more cell cycle generations,
wherein each genetic scratchpad in the one or more genetic scratchpads comprises a polynucleotide sequence and a plurality of target sites within the polynucleotide sequence, and
wherein each of the plurality of molecular changes is associated with a target site among the plurality of target sites;
characterizing, at one or more time points during the time period, a status of molecular changes at each time point for the plurality of target sites in each genetic scratchpad in cells in the cell population, wherein the cells are essentially intact or undisrupted, wherein at least one time point in the one or more time points is two or more cell cycle generations from the beginning of the time period; and
establishing lineage connections or a sequence of molecular changes between cells from different cell cycle generations by comparing statuses of molecular changes of the cells, wherein the molecular changes may represent one or more molecular events.

2. The method of claim 1, wherein said characterizing step further comprises:
applying a set of probes to the cell population,
wherein each probe in the set recognizes and binds to a corresponding target sequence in a target site among the plurality of target sites, and
wherein each probe comprises a label that produces a visible signal upon binding between the probe and its unique target sequence; and
characterizing the of molecular changes status in a plurality of cells in the cell population by detecting the presence or absence of visible signals in the plurality of cells.

3. The method of claim 1, wherein each target site comprises a guide sequence that is recognized by a unique guide molecule, and wherein binding of the unique guide molecule to the guide sequence recruits a molecule that is capable of creating a molecular change at the target site.

4. The method of claim 3, wherein the guide sequence comprises a nucleotide sequence having a length between about 15 nucleic acids to about 80 nucleic acids.

5. The method of claim 3, wherein the guide sequence comprises a nucleotide sequence having a length between about 15 nucleic acids to about 30 nucleic acids.

6. The method of claim 3, wherein the unique guide molecule is a guide RNA (gRNA).

7. The method of claim 3, wherein the molecule is a nuclease, recombinase or integrase.

8. The method of claim 7, wherein the nuclease is Cas9 nuclease

9. The method of claim 1, wherein the multiple time points during the time period cover two or more cell cycle generations.

10. The method of claim 1, wherein the multiple time points during the time period cover three or more cell cycle generations.

11. The method of claim 1, wherein the multiple time points during the time period cover five or more cell cycle generations.

12. The method of claim 1, wherein the plurality of molecular changes comprises a plurality of mutations.

13. The method of claim 12, wherein the plurality of mutations comprises one selected from the group consisting of an insertion mutation, a deletion mutation, a point mutation, multiple point mutations, and combinations thereof.

14. The method of claim 3, wherein each target site further comprises a barcode sequence linked to the guide sequence.

15. The method of claim 14, wherein the barcode sequence comprises a nucleotide sequence having a length between about 400 nucleic acids to about 2,000 nucleic acids.

16. The method of claim 14, wherein the barcode sequence comprises a nucleotide sequence having a length between about 50 nucleic acids to about 200 nucleic acids.

17. The method of claim 1, wherein each target site in a plurality of target sites within at least one genetic scratchpad comprises the same guide sequence that is recognized by a unique guide molecule.

18. The method of claim 1, wherein each target site in a plurality of target sites within at least one genetic scratchpad comprises a different guide sequence that is recognized by a unique and different guide molecule.

19. The method of claim 18, wherein the plurality of target sites within at least one genetic scratchpad comprises one selected from the group consisting of two or more different guide sequences, three or more different guide sequences, five or more different guide sequences, eight or more different guide sequences, 10 or more different guide sequences, 15 or more different guide sequences, 20 or more different guide sequences, and 30 or more different guide sequences.

20. The method of claim 1, wherein the characterizing step further comprises:
applying a set of probes to cells in the cell population, wherein each probe comprises a nucleic acid sequence designed to bind to a target site within the plurality of target site, and wherein each probe is associated with a label that produces a signal upon binding between the probe and its corresponding target site;
characterizing a mutation status at the plurality of target sites based on the absence and presence of signals, wherein absence of a signal indicates a mutation at the target site and the presence of a signal indicates an intact target site, or vice versa.

21. The method of claim 20, wherein the set of probes comprises RNA probes or DNA probes.

22. The method of claim 20, wherein probes in the set of probes are associated with multiple labels that produce different signals.

23. The method of claim 20, wherein each probe of the set of probes is designed to bind to a guide sequence within a target site within the plurality of target site.

24. The method of claim 23, wherein each probe of the set of probes is designed to further bind to a barcode sequence linked to the guide sequence within a target site within the plurality of target site.

25. A system for characterizing lineage information or molecular events among cells in a cell population, comprising:
a housing component for one or more cells in a cell population, wherein a plurality of molecular changes is introduced over a time period of multiple cell cycle generations in at least one of one or more genetic scratchpads in one or more cells in a cell population,
wherein the cell population comprises cells that have developed for one or more cell cycle generations,
wherein each genetic scratchpad in the one or more genetic scratchpads comprises a polynucleotide sequence and a plurality of target sites within the polynucleotide sequence, and
wherein each of the plurality of molecular changes is associated with a target site among the plurality of target sites;
a characterization component, configured to characterize the cell population, at one or more time points during the time period, a status of molecular events at each time point for the plurality of target sites in each genetic scratchpad in cells in the cell population, wherein the cells are essentially intact or undisrupted, wherein at least one time point in the one or more time points is two or more cell cycle generations from the beginning of the time period; and
an analytical component, designed to receive data from the characterization component and establish lineage connections or a sequence of molecular changes between cells from different cell cycle generations by comparing statuses of molecular changes of the cells, wherein the molecular changes may represent one or more molecular events.
